(19) United States
(12) Patent Application Publication
(10) Pub. No.: US 2011/0092490 A1
(43) Pub. Date: Apr. 21, 2011

(54) PYRIMIDINES, TRIAZINES AND THEIR USE AS PHARMACEUTICAL AGENTS

(75) Inventors: Shudong Wang, Nottinghamshire (GB); Shenhua Shi, Nottinghamshire (GB); Andrey Zaytsev, Nottinghamshire (GB); Peter Martin Fischer, Nottinghamshire (GB)

(73) Assignee: THE UNIVERSITY OF NOTTINGHAM, Nottinghamshire (GB)

(21) Appl. No.: 12/934,798

(22) PCT Filed: Mar. 26, 2009
(86) PCT No.: PCT/GB09/50298

§ 371 Date: Dec. 22, 2010

(30) Foreign Application Priority Data
Mar. 26, 2008 (GB) 0805477.7

(51) Int. Cl. A61K 31/5377; C07D 417/14; C07D 417/04; A61K 31/506; A61K 31/496; A61K 31/551; A61P 35/00; A61P 35/02; A61P 17/06; A61P 9/00; A61P 3/00; A61P 37/00; A61P 31/00

(52) U.S. Cl. 514/218; 544/122; 544/331; 544/295; 540/575; 514/235.8; 514/275; 514/252.19

(57) ABSTRACT

A compound of formula (I) and its pharmaceutically acceptable salts or solvates and physiologically hydrolysable, solubilising or immobilisable derivatives wherein: Ar is a 5-membered heteroaryl ring wherein X¹ and X² are one or two heteroatoms or Ar is a 6-membered aromatic ring, wherein heteroatoms are selected from S, O, N, Se; Z is NH, NHCO, NHSO₂, N-alkyl, CH₂NH, CH₂N-alkyl, CH₂, CH₂CH₂, CH═CH, CH₂CONH, SO₂, or SO; Y is N CR³; R¹, R², R⁵, R⁶, R⁷, R⁸ and R⁹ are each independently H, or a substituent; R³, when present, is selected from alkyl and a substituent, with the proviso that when Y is CR³, Ar is a 5-membered heterocycle comprising one or two N heteroatoms and Z is NH, then R3 is selected from C₃₊ alkyl and a substituent; R⁴ is selected from H, alkyl and R¹³ as hereinbefore defined, with the proviso that when R³ is absent, R⁴ is selected from alkyl and a substituent; processes for the preparation thereof, intermediates and precursors therefore and the use thereof as a medicament, and therapeutic compositions comprising the compound.

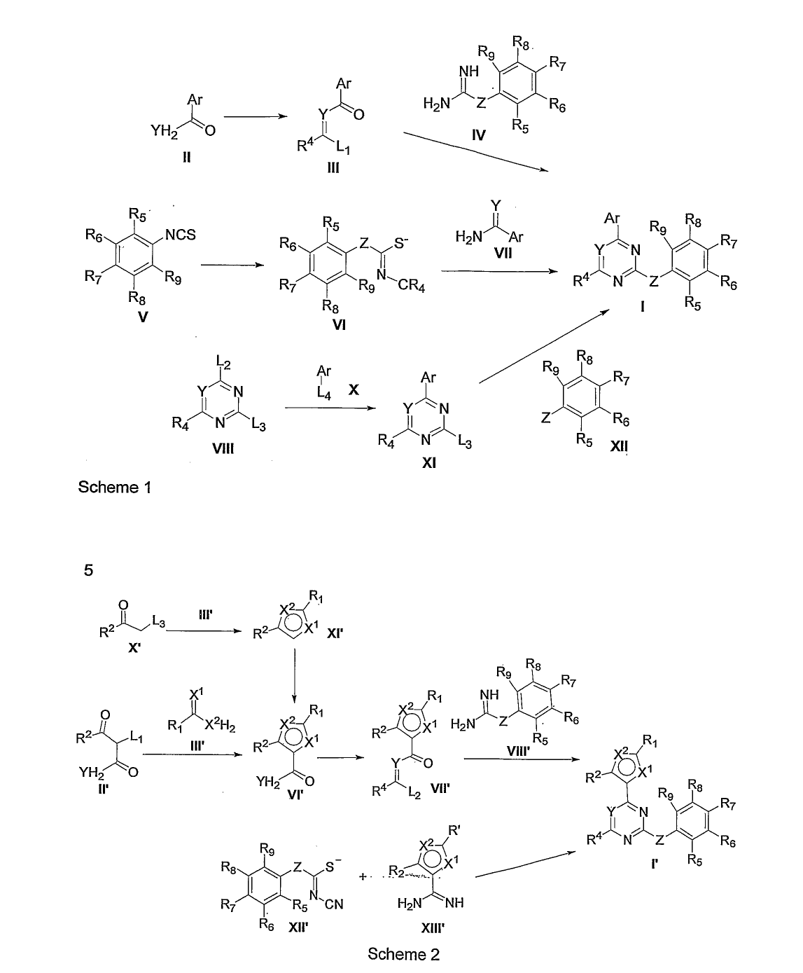

The present invention relates to substituted pyrimidine and [1,3,5]triazine derivatives that have broad therapeutic applications via inhibiting one or more protein kinases. The invention also provides processes for preparing compounds, pharmaceutically acceptable compositions comprising the compounds, and the use of the compounds and methods of using the compounds and compositions in the treatment of various diseases, conditions, or disorders.

### BACKGROUND

The search for new therapeutic agents has been greatly aided in recent years by a better understanding of the structure of enzymes and their biomolecules associated with diseases. One important class of enzymes that has been the subject of extensive study is the protein kinase family.

The protein kinase family is one of the largest in the human genome, comprising 500 genes. The majority of kinases contain a 250-300 amino acid residue catalytic domain with a conserved core structure. This domain comprises a binding pocket for ATP, whose terminal phosphate group transfers covalently to its macromolecular substrates. The protein kinases may be categorized by the substrates they phosphorylate, e.g. protein-serine/threonine, protein-tyrosine.

Protein kinases mediate intracellular signalling by effecting a phosphoryl transfer from a nucleoside triphosphate to a protein acceptor that is involved in a signalling pathway. These phosphorylation events are triggered in response to a variety of extracellular and other stimuli and act as molecular on/off switches that can modulate or regulate the target protein biological function. An extracellular stimulus may affect one or more cellular responses related to cell growth, migration, differentiation, secretion of hormones, activation of transcription factors, muscle contraction, glucose metabolism, control of protein synthesis, and regulation of the cell cycle.

Many diseases are associated with abnormal cellular responses triggered by protein kinase-mediated events. These diseases include, but are not limited to allergies and asthma, Alzheimer's disease, autoimmune diseases, bone diseases, cancer, cardiovascular diseases, inflammatory diseases, hormone-related diseases, metabolic diseases, neurological and neurodegenerative diseases. Accordingly, there has been a substantial effort in medicinal chemistry to find protein kinase inhibitors that are effective as therapeutic agents.

A wide variety of molecules capable of inhibiting protein kinase function through antagonising ATP binding are known in the art. We have previously disclosed 2-anilino-4-heteroaryl-pyrimidine compounds with kinase inhibitory properties, particularly against cyclin-dependent kinases (CDKs) (Wang, S.; et al. WO 2003029248, Cyclacel Limited, UK. Fischer, P. M., WO2002079193, Cyclacel Limited, UK. Wang, S.; Fischer, P. M. US2002019404, Cyclacel Limited, UK.; Fischer, P. M.; Wang, S. WO2001072745, Cyclacel Limited, UK) and 2-anilino-4-phenyl-pyrimidine (Wang S., et al. WO2005012262, Cyclacel Limited, UK). Also known are [1,3,5]Triazines with kinase inhibitory properties (Liu C, WO2004032875, Squibb Bristol Myers Co. US; Armistead, D M, et al. WO200125220, Kinetix Pharmaceuticals Inc. US).

Cyclin-dependent kinases (CDKs) are serine/threonine protein kinases that associate with various cyclin subunits, playing pivotal roles in the regulation of cell cycle progression and transcriptional cycle. Ten distinct CDKs (CDK1-9 and 11) are involved in a variety of important regulatory pathways in eukaryotic cells, including cell-cycle control, apoptosis, neuronal physiology, differentiation and transcription.

CDKs may be classified into two major groups, reflecting their functions. The cell cycle regulator CDKs composed primarily of CDK1, CDK2, CDK3, CDK4 and CDK6 function with their cyclin partners including cyclin A, B, D1, D2, D3, E, and F to regulate promotion of the cell cycle. The transcription regulator CDKs, which include CDK7, CDK8, CDK9 and CDK11 work together with cyclin C, H, K, L1, L2, T1 and T2, tend to play roles in transcriptional regulation.

The CDKs have been implicated in cell proliferation disorders, particularly in cancer. Cell proliferation is a result of the direct or indirect deregulation of the cell division cycle and the CDKs play a critical role in the regulation of the various phases of this cycle. Therefore, inhibitors of CDKs and their associated cyclins are useful targets for cancer therapy.

CDKs also play a role in apoptosis and T-cell development, which is predominantly due to the CDK functions in regulation of transcription. For example, clear clinical activity has very recently been obtained in chronic lymphocytic leukaemia (CLL) with CDK inhibitor flavopiridol. CLL is characterised by cellular resistance to apoptosis through up-regulation of anti-apoptotic proteins. Inhibition of transcription at the level of CDK9, which is necessary for mRNA elongation, selectively reinstates apoptosis in CLL cells. There is however a need for pharmacologically and pharmaceutically superior CDK inhibitors with a well-defined kinase selectivity and cellular specificity profile and anti-CLL efficacy, as well as efficacy against other CDK mediated disorders.

Furthermore, numerous viruses require CDKs, particular CDK2, CDK7, and CDK9, for their replication process. CDK inhibitors that restrain viral replication including human immunodeficiency virus, human cytomegalovirus, herpes virus, and varicella-zoster virus have been reported.

Inhibition of CDKs, particular CDK9, is a novel strategy for potential treatment of cardiovascular diseases including cardiohypertrophy. Cardiohypertrophy is characterised by global increases in mRNA and protein synthesis. CDK7 and CDK9 are closely associated with cardiac hypertrophy as they are the main drivers for transcription. Therefore inhibition of CDK9 and its associated cyclins is a relevant drug target for cardiovascular diseases.

Inhibition of CDK is also useful for the treatment of neurodegenerative disorders such as Alzheimer's disease. The appearance of Paired Helical Filaments, associated with Alzheimer's disease, is caused by the hyperphosphorylation of Tau protein by CDK5/p25.

Inhibition of one or more other serine/threonine kinases including the Aurora kinases, Glycogen synthesis kinases (GSKs), polo-like kinases (PLKs) and tyrosine kinases including Ableson tyrosine kinase (BCR-ABL), FMS-related tyrosine kinases (FLT), IkB kinases (IKK), Janus kinases (JAK), platelet-derived growth factor (PDGF) receptor tyrosine kinases, vascular endothelial growth factor (VEGF) receptor tyrosine kinases, and Src family are also useful for the treatment of numerous diseases, conditions or disorders mediated by these kinases.

GSK3 is known to phosphorylate many substrates and is thus involved in the regulation of multiple biochemical pathways. For example, GSK is highly expressed in the central and peripheral nervous systems. GSK3 inhibition is therefore of therapeutic significance in the treatment of CNS disorders such as Parkinsons and Alzheimers diseases.

Furthermore, it has been demonstrated that GSK3 is over-expressed in muscle cells of type II diabetics and that an inverse correlation exists between skeletal muscle GSK3 activity and insulin action. GSK3 inhibition is therefore of therapeutic significance in the treatment of diabetes, particularly type II, and diabetic neuropathy.

Aurora kinases and PLK are also important therapeutic targets for treatment of proliferative disorders. Based on their known functions inhibition of Aurora kinases and PLKs activity should disrupt mitosis leading to cell cycle arrest and therefore slowing tumour growth and induce apoptosis.

The present invention provides a novel class of substituted-2-anilino-4-arylpyrimidines and -4-aryl-[1,3,5]triazin-2-ylphenylamines with broad therapeutic application as protein kinase inhibitors, specifically compounds which are substituted at the 2-, 4-, and 5- and/or 6-positions of pyrimidines or, at the 2-, 4- and 6-positions of [1,3,6]triazines. These compounds have been synthetically difficult to access. We have found that the invention offers a class of compounds which are effective in protein kinase inhibition, offer important benefits in terms of selective inhibition, and are potentially effective therapeutics.

### BRIEF SUMMARY OF THE DISCLOSURE

A first aspect of the present invention relates to a compound of formula I and its pharmaceutically acceptable salts or solvates and physiologically hydrolysable, solubilising or immobilisable derivatives:

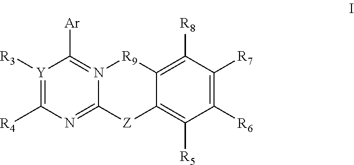

Wherein:

Ar is optionally substituted and is a 5-membered heteroaryl ring wherein X¹ and X² are one or two heteroatoms or Ar is a 6-membered aromatic ring, wherein heteroatoms are selected from S, O, N, Se and wherein optional substituents include R¹ and R²;

Z is NH, NHCO, NHSO₂, N-alkyl, CH₂NH, CH₂N-alkyl, CH₂, CH₂CH₂, CH═CH, CH₂CONH, SO₂, or SO;

Y is N or CR³;

R¹, R², R⁵, R⁶, R⁷, R⁸ and R⁹ are each independently H, alkyl, or R¹³ wherein R¹³ is selected from R¹⁰, alkyl-R¹⁰, aryl, heteroaryl and combinations of two or more thereof and combinations with one or more alkyl and R¹¹, or R¹³ is one or more moieties R¹⁴ selected from O-, N-, NH-, CO-, COO-, CON-, CONH-, SO₂-, SO₂N-, SO₂NH-linking one or more alkyl, aryl, heteroaryl or R¹⁰ or R¹¹ groups or combinations thereof, directly or via a moiety selected from alkylene, arylene, heteroarylene or combination thereof, wherein alkyl, aryl, heteroaryl groups or moieties thereof may be substituted with one or more groups R¹⁵ selected from halogeno, NH₂, NO₂, CN, OH, COOH, CONH₂, C(═NH)NH₂, SO₃H, SO₂NH₂, SO₂CH₃, OCH₃, CF₃ or R¹³ is selected from a group R¹⁵;

or two of R⁵ to R⁹ are linked to form a cyclic ether containing one or more oxygen atoms;

R³, when present, is selected from alkyl and R¹³ as hereinbefore defined, with the proviso that when Y is CR³, Ar is a 5-membered heterocycle comprising one or two N heteroatoms and Z is NH, then R³ is selected from C₃₊ alkyl and R¹³ as hereinbefore defined;

R⁴ is selected from H, alkyl and R¹³ as hereinbefore defined, with the proviso that when R³ is absent, R⁴ is selected from alkyl and R¹³ as hereinbefore defined;

wherein at least one of R¹, R², R⁴, R⁵, R⁶, R⁷, R⁸ and R⁹ and R³ or R¹² where present, comprise a group R¹⁰ or R¹¹ wherein R¹⁰ and R¹¹ comprise one or more solubilising moieties chosen from i) neutral hydrophilic groups, ii) ionisable organic acids, iii) ionisable organic bases and combinations thereof.

A further aspect of the invention relates to a compound of formula I wherein at least one of R¹⁰ or R¹¹ further comprises an immobilising moiety chosen from iv) chemical functions or moieties providing covalent or non-covalent attachment or binding to a solid phase or an immobile receptor.

Further aspects of the invention relate to a process for the preparation of a compound of formula I as hereinbefore defined, to a process for the preparation of precursors or intermediates, and to novel precursors or intermediates.

In a further aspect the invention relates to the use of a compound of formula I or a pharmaceutically acceptable salt, solvate or physiologically hydrolysable, solubilising or immobilising derivative thereof, in the manufacture of a medicament for treating a condition mediated by an enzyme selected from one or more CDK, aurora kinase, GSK, PLK, BCR-ABL, FLT, IKK, JAK, PDGF or VEGF and Src family enzymes, particularly from one or more CDK2, CDK7, CDK8, CDK9, CDK11, GSK-3, aurora kinase, PLK or at least one tyrosine kinase.

In a further aspect of the invention, there is provided a method for treating a condition mediated by one or more enzymes selected from CDK, aurora kinase, GSK, PLK, BCR-ABL, FLT, IKK, JAK, PDGF or VEGF and Src family enzymes, particularly from one or more CDK2, CDK7, CDK8, CDK9, CDK11, GSK-3, aurora kinase, PLK or tyrosine kinase enzyme, in a human or animal subject, the method comprising administering to a human or animal in need thereof a therapeutically effective amount of a compound of formula I or a pharmaceutically acceptable salt, solvate, or physiologically hydrolysable, solubilising or immobilising derivative thereof.

In a further aspect of the invention, there is provided the use of a compound of formula I or a pharmaceutically acceptable salt, solvate, or physiologically hydrolysable, solubilising or immobilising derivative thereof in a method for treating a condition mediated by an enzyme selected from one or more CDK, aurora kinase, GSK, PLK, BCR-ABL, FLT, IKK, JAK, PDGF or VEGF and Src family enzymes, particularly from one or more CDK2, CDK7, CDK8, CDK9, CDK11, GSK-3, aurora kinase, PLK or tyrosine kinase.

A further aspect of the invention relates to the use of a compound of formula I or a pharmaceutically acceptable salt or solvate or physiologically hydrolysable, solubilising or immobilising derivative thereof, in an assay for identifying candidate compounds capable of treating a condition mediated by an enzyme selected from one or more CDK, aurora kinase, GSK, PLK, BCR-ABL, FLT, IKK, JAK, PDGF or VEGF and Src family enzymes, particularly from one or more CDK2, CDK7, CDK8, CDK9, CDK11, GSK-3, aurora kinase, PLK or tyrosine kinase.

A further aspect of the invention relates to a pharmaceutical composition comprising a compound of formula I or a pharmaceutically acceptable salt or solvate, or physiologically hydrolysable, solubilising or immobilising derivative thereof, in association with one or more diluents, carriers or excipients.

### BRIEF DESCRIPTION OF THE DRAWINGS

Embodiments of the invention are further described hereinafter with reference to the accompanying drawings, in which Schemes 1 and 2 illustrate processes for preparing compounds of the invention.

### DETAILED DESCRIPTION

As used herein the term “alkyl” includes both straight chain and branched alkyl groups. The alkyl group May be substituted (mono- or poly-) or unsubstituted. Suitable substituents include, for example, halo, CF₃, OH, CN, NO₂, SO₃H, SO₂NH₂, SO₂Me, NH₂, COOH, CONH₂ and alkoxy. Preferably, the alkyl group is a C₁₋₂₀ alkyl group, more preferably a C₁₋₁₅, more preferably still a C₁₋₁₂ alkyl group, more preferably still, a C₁₋₆ alkyl group, more preferably a C₁₋₃ alkyl group. Particularly preferred alkyl groups include, for example, methyl, ethyl, propyl, isopropyl, butyl, isobutyl, tert-butyl, pentyl and hexyl.

As used herein, the term “heteroalkyl” includes an alkyl group as defined above which comprises one or more heteroatoms.

As used herein, the term “cycloalkyl” refers to a cyclic alkyl group which may be substituted (mono- or poly-) or unsubstituted. Suitable substituents include, for example, halo, CF₃, OH, CN, NO₂, SO₃H, SO₂NH₂, SO₂Me, NH₂, COOH, CONH₂ and alkoxy.

Likewise, the term “cycloheteroalkyl” refers to a cyclic heteroalkyl group which may be substituted (mono- or poly-) or unsubstituted. Suitable substituents include, for example, halo, CF₃, OH, CN, NO₂, SO₃H, SO₂NH₂, SO₂Me, NH₂, COOH, CONH₂ and alkoxy. Preferred cycloheteroalkyl groups include morpholino, piperazinyl and piperidinyl groups.

As used herein, the term “aryl” refers to an aromatic, substituted (mono- or poly-) or unsubstituted group, and includes, for example, phenyl, naphthyl etc. Again, suitable substituents include, for example, halo, CF₃, OH, CN, NO₂, SO₃H, SO₂NH₂, SO₂Me, NH₂, COOH, CONH₂ and alkoxy.

As used herein, the term “heteroaryl” refers to an aromatic, substituted (mono- or poly-) or unsubstituted group, which comprises one or more heteroatoms. Preferred heteroatoms include N, S, O. Preferred heteroaryl groups include pyrrole, pyrazole, pyrimidine, pyrazine, pyridine, quinoline, triazine, triazole, thiophene, selenazol, thiazole and furan. Again, suitable substituents include, for example, halo, CF₃, OH, CN, NO₂, SO₃H, SO₂NH₂, SO₂Me, NH₂, COOH, CONH₂ and alkoxy.

As used herein the term “halo” or “halogeno” refers to F, Cl, Br or I.

In a first embodiment of the invention there is provided a compound of formula I′

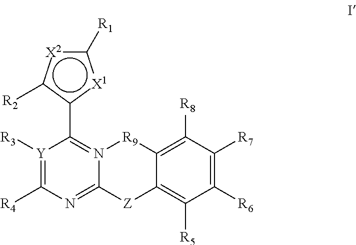

wherein X¹ and X² are each independently selected from NH or N, O, S, Se, CH and C

R¹⁵ and at least one of X¹ and X² is selected from NH or N, O, S and Se, and wherein R¹⁵ is as hereinbefore defined for R¹, and all other variables are as hereinbefore defined.

Preferably there is provided a compound of formula I′ wherein:

one of X¹ and X² is CH or CR¹⁵, and the other of X¹ and X² is S, O, NH, NR¹⁵, or Se; or

one of X¹ and X² is S, O or Se, and the other of X¹ and X² is N; or

one of X¹ and X² is N, and the other of X¹ and X² is NH or NR¹⁵; and wherein all other variables are as hereinbefore defined.

More preferably X¹ is S and X² is N or X² is S and X¹ is N.

In an alternative embodiment of the invention there is provided a compound of formula I″

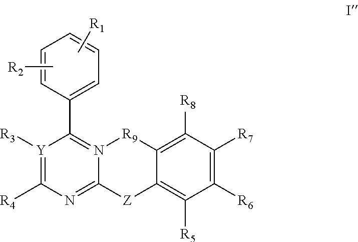

wherein all variables are as hereinbefore defined.

More preferably, a compound of formula I′ or I″ comprises a mono- or di-substituted phenyl, thiazol-4-yl, thiazol-5-yl, imidazol-4-yl, imidazol-5-yl, pyrrol-4-yl or pyrrol-5-yl group attached to the pyrimidine or [1,3,5]triazine ring through one of the ring carbon atoms; most preferably a phenyl, thiazol-4-yl or thiazol-5-yl group.

Where R¹⁰ or R¹¹ comprises a neutral hydrophilic group (i) as hereinbefore defined, this preferably includes groups containing mono-, di- and polyhydroxylated saturated or unsaturated aliphatic, alicyclic or aromatic systems, carbohydrate derivatives, ethers and polyethers optionally containing one or more hydroxyl groups, O- and/or S-containing heterocyclic systems optionally containing one or more hydroxyl groups, aliphatic or aromatic systems containing a carboxamide, sulfoxide, sulfone, or sulfonamide function, and halogenated alkylcarbonyl groups.

Where R¹⁰ or R¹¹ comprises an ionisable organic acid (ii) as hereinbefore defined, this preferably includes groups comprising one or more of the functions COOH, SO₃H, OSO₃H, PO₃H₂, and OPO₃H₂.

Where R¹⁰ or R¹¹ comprises an ionisable basic group (iii) as hereinbefore defined, this preferably includes aliphatic, alicyclic, aromatic, or heterocyclic groups comprising one or more of the functions —O—, —NH₂, —NH—, ═N—, quarternary amine salts, guanidine, and amidine, optionally substituted by one or more substituents selected from halogen, SO₂alkyl, alkyl optionally substituted by one or more OH or halogen groups, CHO, COalkyl, aralkyl, COOalkyl and an ether group substituted by one or more OH groups.

In one embodiment R¹⁰ and R¹¹ may consist of natural or unnatural amino acid residues and peptides, or their derivatives.

Preferably R¹⁰ or R¹¹ is selected from

v) OSO₃H, PO₃H₂, OPO₃H₂;

vi) Y′ where Y′ is selected from aliphatic, alicyclic, aromatic, or heterocyclic groups comprising one or more of the functions —O—, —NH₂, —NH—, ═N—, amidine, optionally substituted by one or more substituents selected from halogen, SO₂alkyl, alkyl optionally substituted by one or more OH or halogen groups, COalkyl, aralkyl, COOalkyl and an ether group substituted by one or more OH groups;

(vii) NHCO(CH₂)ₘ[NHCO(CH₂)_{m′}]ₚ[NHCO(CH₂)_{m′}]_{q}Y′ or NHCO(CH₂)ₜNH(CH₂)_{t′}Y′ where p and q are each 0 or 1, and m, m′; m″, t and t′ are each independently an integer from 1 to 10; and

(viii) (CH₂)ₙNR¹⁹COR¹⁷, (CH₂)_{n′}NR²⁰SO₂R¹⁸, or SO₂R²¹, where R¹⁷, R¹⁸ and R²¹ are each alkyl groups optionally comprising one or more heteroatoms, and which are optionally substituted by one or more substituents selected from OH, NH₂, halogen and NO₂, R¹⁹ and R²⁰ are each independently H or alkyl, and n and n′ are each independently 0, 1, 2, or 3;

(ix) an ether or polyether optionally substituted by one or more hydroxyl groups or one or more Y′ groups;

(x) (CH₂)ᵣNH₂; where r is 0, 1 , 2, or 3;

(xi) (CH₂)_{r′}OH; where r′ is 0, 1 , 2, or 3;

(xii) (CH₂)_{n″}NR²²COR²³ where R²² is H or alkyl, n″ is 0, 1, 2 or 3 and R²³ is an aryl or heteroaryl group, each of which may be optionally substituted by one or more substituents selected from halogeno, NO₂, OH, alkoxy, NH₂, COOH, CONH₂ and CF₃;

(xiii) SO₂NR²⁴R²⁵ where R²⁴ and R²⁵ are each independently H, alkyl, aralkyl, CO-alkyl or aryl, with the proviso that at least one of R²⁴ and R²⁵ is other than H, or R²⁴ and R²⁵ are linked to form a cyclic group optionally containing one or more heteroatoms selected from N, O and S, and wherein said alkyl, aryl or cyclic group is optionally substituted by one or more substituents selected from halogeno, NO₂, OH, alkoxy, NH₂, COOH, CH₂CO₂-alkyl, CONH₂ and CF₃;

(xiv) N-piperidinyl, piperidinyl, N-piperazinyl, N-diazepanyl, N-pyridinyl, N-pyrrolidinyl, N-morpholinyl or N-thiomorpholinyl, each of which may be optionally substituted by one or more alkyl, alkoxy, aryl, CHO or CO-alkyl groups.

In one preferred embodiment of the invention, each R¹⁰ or R ¹¹ is independently selected from a C₁₋₃₀ hydrocarbyl group, optionally comprising up to twelve heteroatoms selected from N, S, and O, and optionally bearing up to six substituents each independently selected from a group R¹⁵ as hereinbefore defined or comprising a moiety R¹⁴ as hereinbefore defined, and a group R¹⁵.

Preferably a compound of formula I as hereinbefore defined bears up to six substituents selected from R¹ to R⁹, R¹² and R¹⁶ as hereinbefore defined each comprising one or more heteroatoms selected from N, S, and O, and alternatively or additionally each comprising one or more moieties R¹⁴ or groups R¹⁵ as hereinbefore defined, wherein the combined substituents comprise up to ten heteroatoms or atoms N, S and O.

Preferably Z is NH or NR¹⁶.

Preferably Y is N or CR³.

Preferably R¹³ is selected from alkyl-R¹⁰, alkyl-cycloalkyl which may be part unsaturated, alkyl-cycloheteroalkyl, aryl, aryl-R¹⁰, aralkyl, aralkyl-R¹⁰, alkyl-heteroaryl, halogeno, NO₂, CN, OH, O-alkyl, O-cycloalkyl which may be part unsaturated, O-aryl, O-heteroaryl, O—R¹⁰, NH₂, NH-alkyl, part unsaturated NH-cycloalkyl, NH-cycloheteroalkyl, NH-aryl, NH-heteroaryl, N-(alkyl)₂, N-(aryl)₂, N-(alkyl)(cycloalkyl), N-(alkyl)(cycloheteroalkyl), N-(alkyl)(aryl), N-(alkyl)(heteroaryl), NH—R¹⁰, N—(R¹⁰)(R¹¹), N-(alkyl)(R¹⁰), N-(aryl)(R¹⁰), COOH, COO—R¹⁰, CONH₂, CONH-alkyl, CONH-aryl, CONH-heteroaryl, CON-(alkyl)(R¹⁰), CON(aryl)(R¹⁰), CON(heteroaryl)(R¹⁰), CONH—R¹⁰, CON—(R¹⁰)(R¹¹), NHCO-alkyl, NHCO-aryl, NHCO-heteroaryl, NHCO—R¹⁰, SO₃H, SO₂-alkyl, SO₂-alkyl-R¹⁰, SO₂-aryl, SO₂-aryl-R¹⁰, SO₂-heteroaryl, SO₂-heteroaryl-R¹⁰, SO₂NH₂, SO₂NH—R¹⁰, SO₂N—(R¹⁰)(R¹¹), NHSO₂R¹⁰, CF₃, CO—R¹⁰, CO-alkyl, CO-alkyl-R¹⁰, CO-cycloheteroalkyl, CO-aryl, CO-aryl-R¹⁰, CO-heteroaryl, CO-heteroarylalkyl or R¹⁰, wherein alkyl, aryl, aralkyl, heteroaryl groups may be further substituted with one or more groups selected from halogeno, NO₂, CN, OH, O-methyl, NH₂, COOH, CONH₂ and CF₃. Preferably a cycloheteroalkyl is a morpholino, piperazinyl or piperadinyl.

Preferably R¹ is selected from NH-alkyl, part unsaturated NH-cycloalkyl, NH-heteroaryl, O-alkyl, O-cycloalkyl which may be part unsaturated, O-heteroaryl, alkyl-heteroaryl, alkyl-cycloalkyl which may be part unsaturated.

Preferably R² is selected from H, alkyl, such as C₁₋₅-alkyl, aryl, NH-alkyl, part unsaturated NH-cycloalkyl, NH-heteroaryl, O-alkyl, O-cycloalkyl which may be part unsaturated, O-heteroaryl, alkyl-heteroaryl and alkyl-cycloalkyl which may be part unsaturated.

Preferably R⁵ is selected from H, O-alkyl, particularly OCH₃, CF₃, alkyl or halogeno.

Preferably R⁶ and R⁸ are independently selected from a sulphonyl, carbonyl, amide or sulphonamide, or thioether link to an unsubstituted or substituted 6 membered cyclic or heterocyclic, or aromatic or heteroaromatic ring, wherein substituents are as hereinbefore defined. More preferably R⁶ and R⁸ are independently selected from SO₂-cycloheteroalkyl, SO₂-cycloalkyl, SO₂-heteroaryl, SO-cycloheteroalkyl, SO-cycloalkyl, SO-heteroaryl, CO-cycloheteroalkyl, CO-cycloalkyl, CO-heteroaryl, N-(alkyl)(cycloalkyl), N-(alkyl)(cycloheteroalkyl), or N-(alkyl)(heteroaryl) more preferably wherein the cycloheteroalkyl is heteroatom linked and may be unsubstituted or substituted comprising one, two or three heteroatoms selected from N, O, S. More preferably a cycloheteroalkyl is a N-alkyl-morpholino, N-alkyl-piperazine or N-alkyl-piperadine. Most preferably R⁶ and R⁸ are independently selected from N-linked N-(alkyl)(cycloheteroalkyl), SO₂-cycloheteroalkyl and CO-cycloheteroalkyl most preferably such as N-(alkyl)(morpholino), N-(alkyl)(piperazine), N-(alkyl)(piperadine), SO₂-piperazines, SO₂-morpholines, CO-piperazines, CO-morpholines, CO-piperadine or the like.

Preferably R⁷ is selected from alkyl, for example C₁₋₅ alkyl, CONH₂, CONH-alkyl, CN, OH, CF₃, O-alkyl, halogeno, NH₂, NH-alkyl and NHCO-alkyl.

Preferably R⁹ is selected from H, halogeno, O-alkyl, more preferably H, halogeno and O—C₁₋₅ alkyl.

Preferably R³ is selected from C₁₋₆ alkyl, more preferably, i-propyl, i-butyl or t-butyl, or R¹³ as hereinbefore defined. More preferably R³ is selected from C₄₊ alkyl and R¹³ as hereinbefore defined, or from R¹³ as hereinbefore defined. More preferably R³ is selected from CN, CF₃, halogeno, NO₂, NH₂, NH-alkyl, N-(alkyl)(R¹⁰), NH-cycloheteroalkyl, NHSO₂R¹⁰, CONH₂, CONH-(alkyl), CON-(alkyl)(R¹⁰), R¹⁰, CO-cycloheteroalkyl, CO-heteroaryl, CONH-heteroaryl, CH₂-cycloheteroalkyl, CH₂-heteroaryl, cycloheteroalkyl, heteroaryl, and C₂₋₆ or C₄₋₆ alkyl, wherein alkyl, cycloheteroalkyl, aryl, aralkyl, heteroaryl groups may be further substituted with one or more groups selected from halogeno, NO₂, CN OH, O-methyl, NH₂, COOH, CONH₂ and CF₃.

Preferably R⁴ is selected from alkyl and R¹³ as hereinbefore defined; more preferably R⁴ is selected from amino, halogeno, such as Cl, and alkyl.

Preferably up to six of R¹, R², R³, R⁴, R⁵, R⁶, R⁷, R⁸, R⁹, R¹² and R¹⁶, for example one, two, three or four thereof, shall correspond to or contain one or more of the group R¹⁰ or R¹¹. Preferably either or both of R³ and R⁴ comprise or contain one or more of the group R¹⁰ or R¹¹. Two or more groups R¹⁰ and/or R¹¹ may be the same or different.

Preferably R¹, R², R³, R⁵ or R⁷ comprises or contains a solubilising moiety R¹⁰ or R¹¹.

One preferred embodiment of the invention relates to a compound of formula wherein:

one of X¹ and X² is selected from S, O, NH, NR¹⁵ and Se and the other thereof is N.

Y is CR³ or N;

Z is NH;

each R¹³ is alkyl-R¹⁰, alkyl-cycloalkyl which may be part unsaturated, alkyl-cycloheteroalkyl, aryl, aryl-R¹⁰, aralkyl, aralkyl-R¹⁰, alkyl-heteroaryl, halogeno, NO₂, CN, OH, O-alkyl, O-cycloalkyl which may be part unsaturated, O-aryl, O-heteroaryl, O—R¹⁰, NH₂, NH-alkyl, part unsaturated NH-cycloalkyl, NH-cycloheteroalkyl, NH-aryl, NH-heteroaryl, N-(alkyl)₂, N-(aryl)₂, N-(alkyl)(cycloalkyl), N-(alkyl)(cycloheteroalkyl), N-(alkyl)(aryl), N-(alkyl)(heteroaryl), NH—R¹⁰, N—(R¹⁰)(R¹¹), N-(alkyl)(R¹⁰), N-(aryl)(R¹⁰), COOH, COO—R¹⁰, CONH₂, CONH-alkyl, CONH-aryl, CONH-heteroaryl, CON-(alkyl)(R¹⁰), CON(aryl)(R¹⁰), CON(heteroaryl)(R¹⁰), CONH—R¹⁰, CON—(R¹⁰)(R¹¹), NHCO-alkyl, NHCO-aryl, NHCO-heteroaryl, NHCO—R¹⁰, SO₃H, SO₂-alkyl, SO₂-alkyl-R¹⁰, SO₂-aryl, SO₂-aryl-R¹⁰, SO₂-heteroaryl, SO₂-heteroaryl-R¹⁰, SO₂NH₂, SO₂NH—R¹⁰, SO₂N—(R¹⁰)(R¹¹), NHSO₂R¹⁰, CF₃, CO—R¹⁰, CO-alkyl, CO-alkyl-R¹⁰, CO-cycloheteroalkyl, CO-aryl, CO-aryl-R¹⁰, CO-heteroaryl, CO-heteroarylalkyl or R¹⁰, wherein alkyl, aryl, aralkyl, heteroaryl groups may be further substituted with one or more groups selected from halogeno, NO₂, CN, OH, O-methyl, NH₂, COOH, CONH₂ and CF₃.

More preferably R⁴ is amino, halogeno, or alkyl;

More preferably R⁵ is O-alkyl, CF₃, alkyl, or halogeno;

More preferably each R⁶ or R⁸ is independently SO₂-cycloheteroalkyl, SO₂-heteroaryl, SO-cycloheteroalkyl, SO-heteroaryl, CO-cycloheteroalkyl or CO-heteroaryl; preferably, the cycloheteroalkyl group is a N-alkyl-morpholino, N-alkylpiperazine, N-alkylpiperadine;

More preferably R⁷ is alkyl, CN, OH, CF₃, O-alkyl, halogeno, NH₂, CONH—R¹⁰, NHR¹⁰, or NHCO—R¹⁰.

More preferably a solubilising moiety R¹⁰ or R¹¹ corresponds to or is contained within R¹, R², R³ or R⁵ and R⁹ is H.

Especially preferred compounds of the invention are those of formula I′ wherein one X¹ or X² is S, another X¹ or X² is N, Y is CR³ or N, Z is NH, R¹ and R² are amino, alkyl, heteroaryl or aryl, R³ is C₁₋₄ alkyl, CN, CF₃, halogeno, NO₂ O-alkyl, NH₂, NH-alkyl, N(alkyl)₂, CO₂alkyl, CO-alkyl, CONH₂, CONH-alkyl or heteroaryl, R⁴ is amino, halogeno or alkyl; R⁵ is OMe, alkyl, or halogeno, each R⁶ or R⁸ is independently SO₂-cycloheteroalkyl, SO-cycloheteroalkyl, SO₂-heteroaryl, SO-heteroaryl, CO-cycloheteroalkyl or CO-heteroarylalkyl, R⁷ is alkyl, OH, CF₃, O-alkyl, halogeno, or NH₂ and the solubilising moiety corresponds to or is contained within R¹, R², R³ or R⁵; and wherein R⁹ is H.

Especially preferred compounds of the invention are those of formula I″ wherein:

Z is NH;

R¹ and R² are amino, alkyl, or aryl or R² is H; more preferably R¹ and R² are selected from NH-alkyl, part unsaturated NH-cycloalkyl, NH-heteroaryl, O-alkyl, O-cycloalkyl which may be part unsaturated, O-heteroaryl, alkyl-heteroaryl, alkyl-cycloalkyl which may be part unsaturated; or R² is H, alkyl, such as C₁₋₅-alkyl or aryl, such as C₆ aryl;

R³ is CN, CONH-alkyl, CF₃, halogeno, NO₂, heteroaryl or is contained with R¹³;

R⁴ is amino, halogeno or alkyl;

R⁵ is OMe, alkyl, or halogeno;

each R⁶ and R⁸ is independently selected from SO₂-cycloheteroalkyl, SO-cycloheteroalkyl, CO-cycloheteroalkyl and CO-heteroaryl;

R⁷ is alkyl, OH, CF₃, O-alkyl, halogeno, or NH₂;

R⁹ is H; and the solubilising moiety corresponds to or is contained within R¹, R², R³ or R⁵.

Compounds of the invention include compounds of formula I′:

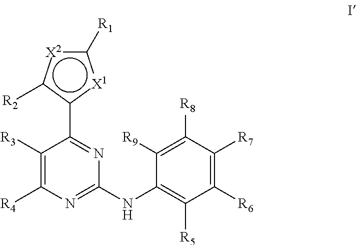

as shown in Table 1 wherein X¹═S, X²═N

| Cpd | R₁ | R₂ | R₃ | R₄ | R₅ | R₆ | R₇ | R₈ | R₉ |
|---|---|---|---|---|---|---|---|---|---|
| 1.1 | NHCH₃ | CH₃ | CN | H | H | “MS” | CH₃ | H | H |
| 1.2 | NHCH₃ | CH₃ | CN | H | H | H | OH | H | H |
| 1.3 | NHCH₃ | CH₃ | CN | H | H | OH | H | H | H |
| 1.4 | NHCH₃ | CH₃ | CN | H | H | “MC” | H | H | H |
| 1.5 | NHCH₃ | CH₃ | CN | H | H | “AcPzC” | H | H | H |
| 1.6 | NHCH₃ | CH₃ | CN | H | H | COOH | H | H | H |
| 1.7 | NHCH₃ | CH₃ | CN | H | H | NO₂ | H | H | H |
| 1.8 | NHCH₃ | CH₃ | CN | H | H | H | SO₂NH₂ | H | H |
| 1.9 | NHCH₃ | CH₃ | CN | H | H | SO₂NH₂ | H | H | H |
| 1.10 | NHCH₃ | CH₃ | CN | H | H | “MePzC” | H | H | H |
| 1.11 | NHCH₃ | CH₃ | CN | H | H | H | “M” | H | H |
| 1.12 | NHCH₃ | CH₃ | CN | H | H | “MS” | H | H | H |
| 1.13 | NHCH₃ | CH₃ | CN | H | H | H | “MS” | H | H |
| 1.14 | NHCH₃ | CH₃ | CN | H | H | SO₂CH₃ | H | H | H |
| 1.15 | NHCH₃ | CH₃ | CN | H | H | “PzC” | H | H | H |
| 1.16 | NH₂ | CH₃ | CN | H | H | “MS” | CH₃ | H | H |
| 1.17 | NH₂ | CH₃ | CN | H | H | H | OH | H | H |
| 1.18 | NH₂ | CH₃ | CN | H | H | OH | H | H | H |
| 1.19 | NH₂ | CH₃ | CN | H | CH₃ | H | OH | CH₃ | H |
| 1.20 | NH₂ | CH₃ | CN | H | H | “MC” | H | H | H |
| 1.21 | NH₂ | CH₃ | CN | H | H | “MePzC” | H | H | H |
| 1.22 | NH₂ | CH₃ | CN | H | H | “AcPzC” | H | H | H |
| 1.23 | NH₂ | CH₃ | CN | H | H | “PzC” | H | H | H |
| 1.24 | NH₂ | H | CN | H | H | “MS” | CH₃ | H | H |
| 1.25 | NH₂ | H | NO₂ | H | H | “MS” | CH₃ | H | H |
| 1.26 | NH₂ | H | CONH₂ | H | H | “MS” | CH₃ | H | H |
| 1.27 | NHCH₃ | CH₃ | CN | H | H | “BPzC” | H | H | H |
| 1.28 | NH₂ | CH₃ | CN | H | H | “BPzC” | H | H | H |
| 1.29 | NH₂ | CH₃ | CN | H | H | “MePdCB” | H | H | H |
| 1.30 | NHCH₃ | CH₃ | CN | H | H | “MePdCB” | H | H | H |
| 1.31 | NH₂ | CH₃ | C(═NH)NH₂ | H | H | “MS” | CH₃ | H | H |
| 1.32 | NH₂ | CH₃ | C(═O)NH₂ | H | H | “MS” | CH₃ | H | H |
| 1.33 | NHCH₃ | CH₃ | C(═O)NH₂ | H | H | “MS” | CH₃ | H | H |
| 1.34 | NHCH₃ | CH₃ | C(═NH)NH₂ | H | H | “MS” | CH₃ | H | H |
| 1.35 | “PyEtA” | CH₃ | C(═NH)NH₂ | H | H | “MS” | CH₃ | H | H |
| 1.36 | “PyEtA” | CH₃ | C(═O)NH₂ | H | H | “MS” | CH₃ | H | H |
| 1.37 | “PyMeA” | CH₃ | CN | H | H | “MePzC” | H | H | H |
| 1.38 | “PyMeA” | CH₃ | CN | H | H | “PzC” | H | H | H |
| 1.39 | NH(CH₂)₂CH₃ | CH₃ | CN | H | H | “PzC” | H | H | H |
| 1.40 | NHCH₃ | CH₃ | CN | H | H | (2-hydroxyethyl)“PzC” | H | H | H |
| 1.41 | NHCH₃ | CH₃ | CN | H | H | (2-methoxyethyl)“PzC” | H | H | H |
| 1.42 | NH₂ | CH₃ | CN | H | H | (2-methoxyethyl)“PzC” | H | H | H |
| 1.43 | NH(CH₂)₂CH₃ | CH₃ | CN | H | H | (2-methoxyethyl)“PzC” | H | H | H |
| 1.44 | NH(CH₂)₂CH₃ | CH₃ | CN | H | H | (2-methoxyethyl)“PdC” | H | H | H |
| 1.45 | NH(CH₂)₂CH₃ | CH₃ | CN | H | H | H | “MeDz” | H | H |
| 1.46 | NHCH₂CH₃ | CH₃ | CN | H | H | H | “MeDz” | H | H |
| 1.47 | NHCH₃ | CH₃ | CN | H | H | H | “MeDz” | H | H |
| 1.48 | NH₂ | CH₃ | CN | H | H | H | “MeDz” | H | H |
| 1.49 | NH₂ | CH₃ | CN | H | H | “MeDz” | H | H | H |
| 1.50 | NHCH₃ | CH₃ | CN | H | H | “MeDz” | H | H | H |
| 1.51 | NHCH₂CH₃ | CH₃ | CN | H | H | “MeDz” | H | H | H |
| 1.52 | NH(CH₂)₂CH₃ | CH₃ | CN | H | H | “MeDz” | H | H | H |
| 1.53 | NHCH₃ | CH₃ | CN | H | CH₃ | H | OH | CH₃ | H |

and Table 2 wherein: X¹═N, X²═S

| Cpd | R₁ | R₂ | R₃ | R₄ | R₅ | R₆ | R₇ | R₈ | R₉ |
|---|---|---|---|---|---|---|---|---|---|
| 1.54 | NHCH₃ | H | CN | H | H | “MS” | CH₃ | H | H |
| 1.55 | NHCH₃ | H | NO₂ | H | H | “MS” | CH₃ | H | H |
| 1.56 | NHCH₃ | H | C(═O)NH₂ | H | H | “MS” | CH₃ | H | H |

and compounds of formula I″:

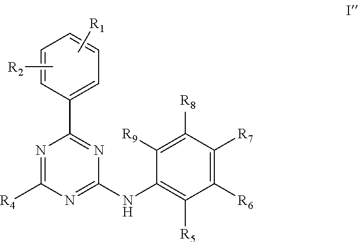

as shown in Table 3

| cpd | R₁ | R₂ | R₄ | R₅ | R₆ | R₇ | R₈ | R₉ |
|---|---|---|---|---|---|---|---|---|
| 2.0 | 3-OCH₃ | H | Cl | H | H | OH | H | H |
| 2.1 | 3-OCH₃ | H | Cl | H | H | OH | H | H |
| 2.2 | 3-OCH₃ | H | Cl | H | NO₂ | H | H | H |
| 2.3 | 3-OCH₃ | H | Cl | H | Br | H | H | H |
| 2.4 | 3-OCH₃ | H | Cl | H | “MS” | CH₃ | H | H |
| 2.5 | 3-OCH₃ | H | NH₂ | H | H | OH | H | H |
| 2.6 | 3-OCH₃ | H | NH₂ | H | NO₂ | H | H | H |
| 2.7 | 3-OCH₃ | H | NHCH₃ | H | NO₂ | H | H | H |
| 2.8 | 3-OCH₃ | H | NHOH | H | NO₂ | H | H | H |
| 2.9 | 3-OCH₃ | H | NH₂ | H | Br | H | H | H |
| 2.10 | 3-OCH₃ | H | NH₂ | H | “MS” | CH₃ | H | H |
| 2.11 | 3-OCH₃ | H | NH₂ | H | “MC” | H | H | H |
| 2.12 | 3-OCH₃ | H | NH₂ | H | “PzC” | H | H | H |

and compounds of formula I′:

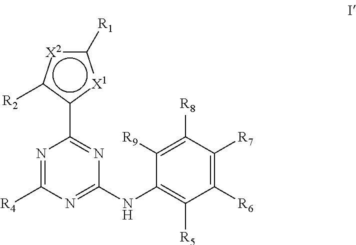

As shown in Table 4 wherein X¹═S, X²═N

| cpd | R₁ | R₂ | R₄ | R₅ | R₆ | R₇ | R₈ | R₉ |
|---|---|---|---|---|---|---|---|---|
| 3.2 | NMeBoc | Ph | Cl | H | NO₂ | H | H | H |
| 3.3 | NMeBoc | Ph | NH₂ | H | NO₂ | H | H | H |
| 3.4 | NHMe | Ph | NH₂ | H | NO₂ | H | H | H |
| 3.5 | NHCH₃ | Ph | NH₂ | H | “MS” | CH₃ | H | H |
| 3.6 | NHCH₃ | tBu | NH₂ | H | “MS” | CH₃ | H | H |
| 3.7 | NHCH₃ | “Py” | NH₂ | H | “MS” | CH₃ | H | H |
| 3.8 | NHCH₃ | CH₃ | NH₂ | H | “MS” | CH₃ | H | H |
| 3.9 | NHCH₃ | CH₃ | NH₂ | H | “MS” | H | H | H |
| 3.10 | NH₂ | CH₃ | NH₂ | H | “MS” | H | H | H |
| 3.11 | NH₂ | Ph | NH₂ | H | “MS” | H | H | H |
| 3.12 | NH₂ | Ph | NH₂ | H | “MS” | CH₃ | H | H |
| 3.13 | NH₂ | Ph | NH₂ | H | “PzS” | H | H | H |
| 3.14 | NH₂ | Ph | NH₂ | H | “MePzS” | H | H | H |
| 3.15 | NH₂ | CH₃ | NH₂ | H | “BPzS” | H | H | H |
| 3.16 | NHCH₃ | CH₃ | NH₂ | H | “BPzS” | H | H | H |
| 3.17 | NHCH₂CH₃ | CH₃ | NH₂ | H | “BPzS” | H | H | H |
| 3.18 | NHCH₂CH₃ | CH₃ | NH₂ | H | “MePzS” | H | H | H |
| 3.19 | NHCH₃ | CH₃ | NH₂ | H | “MePzS” | H | H | H |
| 3.20 | NHCH₃ | CH₃ | NH₂ | H | “PzS” | H | H | H |
| 3.21 | NH₂ | CH₃ | NH₂ | H | “PzS” | H | H | H |
| 3.22 | NH₂ | CH₃ | NH₂ | H | “PzC” | H | H | H |
| 3.23 | NH₂ | CH₃ | NH₂ | H | “MePzC” | H | H | H |
| 3.24 | NHCH₃ | CH₃ | NH₂ | H | “MePzC” | H | H | H |
| 3.25 | NHCH₂CH₃ | CH₃ | NH₂ | H | “MePzC” | H | H | H |
| 3.26 | NHCH₃ | CH₃ | NH₂ | H | “PdC” | H | H | H |
| 3.27 | NHCH₃ | CH₃ | NH₂ | H | “MePdC” | H | H | H |
| 3.28 | NHCH₃ | CH₃ | NH₂ | H | “BPdC” | H | H | H |
| 3.29 | NH₂ | CH₃ | NH₂ | H | “Pz” | H | H | H |
| 3.30 | NH₂ | CH₃ | NH₂ | H | “MePz” | H | H | H |

Wherein

MS=morpholine-4-sulfonyl

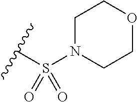

PzC=piperazine-1-carbonyl or piperazin-1-ylmethanone

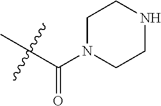

AcPzC=4-Acetylpiperazine-1-carbonyl

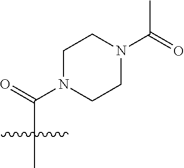

MePzC=4-methylpiperazine-1-carbonyl or 4-methylpiperazin-1-ylmethanone

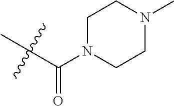

M=morpholino

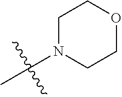

MC=morpholin-4-carbonyl or morpholin-4-yl-methanone

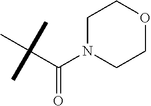

PzS=piperazin-1-ylsulfonyl

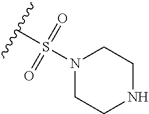

MePzS=4-methylpiperazin-1-ylsulfonyl

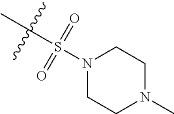

BPzS=4-benzylpiperazin-1-ylsulfonyl

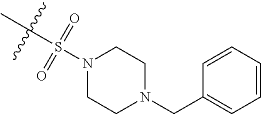

BPzC—benzylpiperazine-1-carbonyl

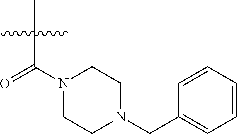

BPdC=1-benzylpiperidin-4-carbonyl or 1-benzylpiperidin-4-ylmethanone

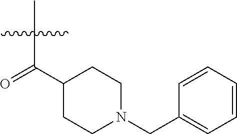

PdC=piperidine-4-carbonyl or piperidin-4-ylmethanone

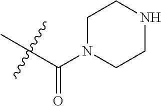

MePdC=1-methylpiperidin-4-ylmethanone or 1-methylpiperidin-4-carbonyl

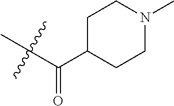

MePdCB—4-(1-methylpiperidine-4carbonyl)benzoyl

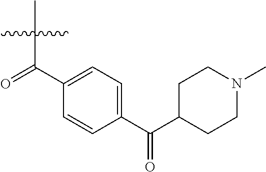

Pz=piperazin-1-yl

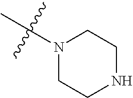

MePz=4-methylpiperazin-1-yl

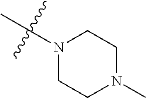

Py=pyridine-3-yl

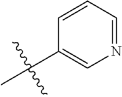

PyEtA=2-(pyridin-3-yl)ethylamino

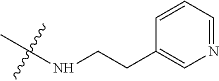

PyMeA=pyridin-3-ylmethylamino

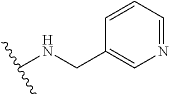

MeDz=4-methyl-1,4-diazepan-1-yl

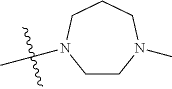

and their pharmaceutically acceptable salts, solvates and physiologically hydrolysable, solubilising or immobilisable derivatives.

In a further aspect of the invention there is provided a compound of formula I as hereinbefore defined wherein one or more R¹⁰ or R¹¹ alternatively or additionally comprise devices for immobilisation thereof. Such devices may be chemical functions that can be used for covalent attachment to solid phases such as functionalised polymers (e.g. agarose, polyacrylamide, polystyrene etc.) as commonly found in matrices (microtitre plate wells, microbeads, membranes, etc.) used for biochemical assays and affinity chromatography. Alternatively, the devices may be small molecules (e.g. biotin) or polypeptides (e.g. antigens), which can be used for non-covalent immobilisation through binding to an immobilised receptor (e.g. avidin or streptavidin in the case of biotin, or a specific antibody in the case of antigens).

In a further aspect of the invention there is provided a precursor to a compound of formula I as hereinbefore defined wherein one or more R¹⁰ or R¹¹ is a solubilising moiety comprising a natural or unnatural amino acid residue, peptide or derivative as hereinbefore defined.

In a further aspect of the invention there is provided a process for the preparation of a compound of formula I as hereinbefore defined. Compounds of formula I may be prepared by any methods known in the art.

Suitably a process for the preparation of a compound of formula I as hereinbefore defined comprises:

(1) reacting a compound of formula III (as illustrated hereinbelow), where Ar is a mono- or di-substituted phenyl, thiazol-4-yl, thiazol-5-yl, imidazol-4-yl, imidazol-5-yl, pyrrol-4-yl or pyrrol-5-yl, preferably Ar is a phenyl, thiazol-4-yl or thiazol-5-yl group, Y is N, or CR³ and L¹ is a leaving group, with a compound of formula IV (as illustrated hereinbelow), where Z and R⁵ to R⁹ are as hereinbefore defined;

or (2) reacting a compound of formula VI (as illustrated hereinbelow) where Z and R⁵ to R⁹ are as hereinbefore defined with a compound of formula VII (as illustrated hereinbelow), where Ar is as hereinbefore defined and Y is N;

or (3) reacting a compound of formula XI (as illustrated hereinbelow) where Y is N or CR³, L³ is any leaving group, preferable halogeno group and Ar and R⁴ are as hereinbefore defined, with a compound of formula XII (as illustrated hereinbelow) where Z and R⁵ to R⁹ are as hereinbefore defined.

Preferably the compound of formula I is a compound of formula I′ as hereinbefore defined, more preferably where Ar is a mono- or di-substituted thiazol-4-yl, thiazol-5-yl, imidazol-4-yl, imidazol-5-yl, pyrrol-4-yl or pyrrol-5-yl attached to the pyrimidine or [1,3,5]triazine ring through one of the ring carbon atoms, most preferably Ar is a thiazol-4-yl or thiazol-5-yl group; or is a compound of formula I″ as hereinbefore defined, more preferably wherein the compounds of formula I″ bear a mono- or di-substituted phenyl attached to the pyrimidine or [1,3,5]triazine ring through one of the ring carbon atoms.

Preferably in process (1) L¹ is any leaving group including N(alkyl)₂, halogen, ester, thioester, more preferably, NMe₂, and the process (1) comprises a variety of methods for example as disclosed in Fischer P M, Wang S. WO 2001072745; and Wang, S.; et al WO 2003029248, Cyclacel Limited, UK and references therein.

Preferably process (2) is conducted via a variety of methods known in the art, particularly, methods described by Liu, C (Liu, C, et al. 2007, Tetrahedron Lett 48, 435) and Hodous, B (Hodous, B. L. J Med Chem, 50, 611).

Preferably in process (3), the compound of formula XI (as illustrated hereinbelow) is obtained by reacting a compound of formula VIII (as illustrated hereinbelow) where L² is any leaving group, preferably a halogeno group, and Y, L³ and R⁴ are as hereinbefore defined, with a compound of formula X (as illustrated hereinbelow), where Ar is as hereinbefore defined and L4 is any boronic acid or derivatives. Palladium-catalysed cross-coupling of VIII (Y is N or CR³) with X or derivatives affords 4-arylated 2-halogeno-pyrimidines or [1.3.6]triazines XI, which are preferably aminated with anilines XII as hereinbefore defined. Alternatively, treatment of VIII with X, and a Grignard reagent such as alkyl magnesium bromide, forms XI which reacts as hereinbefore defined with XII to provide the compounds of formula I.

More preferably the process is as illustrated in Scheme 1 below:

Suitably an alternative process for the preparation of a compound of formula I′ as hereinbefore defined comprises:

(4) condensation reaction between a compound of formula VII′ (as illustrated hereinbelow) wherein R¹, R², R⁴, X¹, X², Y and L² are as hereinbefore defined with a phenylguanidine of formula VIII′ (as illustrated hereinbelow) wherein Z and R⁵ to R⁹ are as hereinbefore defined to obtain pyrimidines or [1,3,5]triazines of formula I′;

or (5) treatment of amidines VI′ (as illustrated hereinbelow) wherein Z and R⁴ to R⁹ are as hereinbefore defined in the presence of POCl₃ followed by alkylation reaction with anilines of formula XII (as illustrated hereinabove) to obtain [1,3,5]triazine triazines of formula I′;

or (6) condensation reaction of a compound of formula XII′ (as illustrated hereinbelow) wherein Z and R⁵ to R⁹ are as hereinbefore defined with an amidine of formula XIII′ (as illustrated hereinbelow) wherein R¹, R², X¹ and X² are as hereinbefore defined, in the presence of base to yield a [1,3,5]triazine of formula I′.

Preferably the compound of formula I′ is as hereinbefore defined, more preferably is a mono- or di-substituted thiazol-4-yl, thiazol-5-yl, imidazol-4-yl, imidazol-5-yl, pyrrol-4-yl or pyrrol-5-yl attached to the pyrimidine or [1,3,5]triazine ring through one of the ring carbon atoms; most preferably is a thiazol-4-yl or thiazol-5-yl group.

Preferably process (4) uses the method described previously (Wang, S. et al. J Med Chem, 2004, 47, 1662-75).

Preferably in process (4) or (5), VII′ may be obtained from the corresponding VI′ by reaction with N,N′-dimethylformamide dimethylacetal (where R⁴═H, L=NMe₂) or tert-butoxy-bis(dimethylamino)methane (Bredereck, H.; et al. Chemische Bernice 1964, 97, (12), 3397).

Preferably ketones VI′ (Y═CH₃) or amides (Y═NH₂) are obtained by cyclisation reaction between II′ (L₁=Cl, Br) with III′ (amides while X¹═O; thioamides while X¹═S, X²═N, R¹=alkyl, NH-alkyl as hereinbefore defined). Compounds VI′ may also be prepared by treatment of ketones X′ with III′ followed by the Friedel-Crafts acylation (Y=CH₃), or amination (Y=NH₂).

Preferably guanidines VIII′ are obtained by reaction of cyanamide or certain of its derivatives using the method of Katritzky, A. R.; et al. Synthetic Communications 1995, 25, 1173.

Preferably in process (6) a compound of formula XIII′ is obtained by reacting phenyl isothiocyanate sodium hydrogen cyanamide to provide N-cyanothiourea XII′.

More preferably the process is as illustrated in Scheme 2 below:

In a further aspect of the invention there are provided novel chemical intermediates of formula I, I′ and I″, IV, VI, VII, XI, XII, VI′, VII′, VIII′, XI′ or XIII′, as hereinbefore defined.

Therapeutic Use

In a further aspect of the invention there is provided the use of one or more compounds of formula I or salts, solvates or derivatives as hereinbefore defined in the manufacture of a medicament for treating a condition mediated by one or more of a CDK, aurora kinase, GSK, PLK and one of Tyrosine kinases as hereinbefore defined, preferably such medicament is capable of inhibiting such enzymes. The compounds of the invention may inhibit any of the steps or stages in the cell cycle.

In one embodiment such medicament is suitable for inhibition of a proliferative disorder mediated by a CDK or PLK, preferably is useful in the treatment of a proliferative disorder, such as cancers, leukaemias and other disorders associated with uncontrolled cellular proliferation such as psoriasis and restenosis, a viral disorder, a cardiovascular disease, a CNS disorder, an autoimmune disease, a bond disease, a hormone-related disease, a metabolic disorder, stroke, alopecia, an inflammatory disease or an infectious disease.

Preferably the compound of formula I is capable of inhibiting one or more of the host cell kinases involved in cell proliferation, viral replication, a cardiovascular disorder, neurodegeneration, autoimmunity, a metabolic disorder, stroke, alopecia, an inflammatory disease or an infectious disease.

A proliferative disorder requires treatment of a susceptible neoplasm and may be selected from the group consisting of chronic lymphocytic leukaemia, lymphoma, leukaemia, breast cancer, lung cancer, prostate cancer, colon cancer, melanoma, pancreatic cancer, ovarian cancer, squamous carcinoma, carcinoma of head and neck, endometrial cancer, and aesophageal carcinoma.

Preferably, the proliferative disorder is a cancer or leukaemia. The term proliferative disorder is used herein in a broad sense to include any disorder that requires control of the cell cycle, for example cardiovascular disorders such as restenosis and cardiomyopathy, auto-immune disorders such as glomerulonephritis and rheumatoid arthritis, dermatological disorders such as psoriasis, anti-inflammatory, anti-fungal, antiparasitic disorders such as malaria, emphysema and alopecia. In these disorders, the compounds of the present invention may induce apoptosis or maintain stasis within the desired cells as required.

As defined herein an effect against a proliferative disorder mediated by a kinase within the scope of the present invention may be demonstrated by the ability to inhibit cell proliferation in an in vitro whole cell assay, for example using any of the cell lines including, but not limiting to A549, A2780, HT29, Saos-2, HCT-116, HeLa, MCF-7, NCI-H460 or by showing inhibition of a CDK enzyme such as CDK1, CDK2, CDK4, CDK5, CDK6, CDK7, CDK8, CDK9, CDK11, or other protein kinases in an appropriate assay. These assays including methods for their performance are described in more detail under Biological Activity.

Studies have shown that human PLKs regulate some fundamental aspects of mitosis. Both PLK1 and PLK2 may have additional post-mitotic functions. Deregulated PLK expressions result in cell cycle arrest and apoptosis. Compounds of the invention are therefore believed to be of use in treating PLK-mediated conditions, particularly proliferative disorders.

A further embodiment relates to the use of compounds of the invention, or pharmaceutically acceptable salts thereof, in the manufacture of a medicament capable of treating a viral disorder mediated by one or more of the host cell CDKs involved in viral replication, i.e. CDK1, CDK2, CDK4, CDK7, CDK8, CDK9 or CDK11 as hereinbefore defined. Preferably such medicament is useful in treating a viral disorder.

Assays for determining CDK activity are described in more detail in the accompanying examples. Using such enzymes assays it may be determined whether a compound is anti-viral in the context of the present invention.

Preferably such medicament is useful in the treatment of viral disorders, such as human cytomegalovirus (HCMV), herpes simplex virus type 1 (HSV-1), human immunodeficiency virus type 1 (HIV-1), and varicella zoster virus (VZV).

Typically such disorder is CDK dependent or sensitive. CDK dependent disorders are associated with an above normal level of activity of one or more CDK enzymes. Such disorders are typically associated with an abnormal, level of activity of CDK1, CDK2, CDK4, CDK7, CDK8, CDK9 and/or CDK11. A CDK sensitive disorder is a disorder in which an aberration in the CDK level is not the primary cause, but is downstream of the primary metabolic aberration. In such scenarios, CDK1, CDK2, CDK4, CDK7, CDK8 CDK9 and/or CDK11 can be said to be part of the sensitive metabolic pathway and inhibitors of these CDKs may therefore be active in treating such disorders.

For use in the treatment of viral disorders, preferably the medicament of the invention is capable of inhibiting CDK2, CDK7, and/or CDK9.

Yet another embodiment relates to the use of compounds of the invention, or pharmaceutically acceptable salts thereof, in the manufacture of a medicament capable of treating cardiovascular diseases mediated by one or more CDKs. Preferably such medicament is useful in treating cardiovascular diseases.

A cardiovascular disease may be selected from the group consisting of ischaemic heart disease (also known as myocardial infarction or angina), hypertension, heart failure, restenosis and cardiomyopathy.

Cardiac hypertrophy is characterised by global increases in mRNA and protein synthesis. CDK9 activity has been demonstrated to be necessary for hypertrophy in cardiomyocytes. Heart-specific activation of CDK9 by cyclin T1 was found to provoke hypertrophy. Compounds of the invention are believed to inhibit CDK9 and are therefore believed to be of use in the prevention and treatment of cardiac hypertrophy.

Yet another embodiment relates to the use of a compound of the invention in the manufacture of a medicament capable of treating neurodegenerative disorders mediated by one or more GSKs or CDKs. Preferably such medicament is useful in the treatment of neurodegenerative disorders such as Alzheimer's disease.

Tau is a GSK-3 substrate which has been implicated in the etiology of Alzheimer's disease. In healthy nerve cells, Tau co-assembles with tubulin into microtubules. However, in Alzheimer's disease, tau forms large tangles of filaments, which disrupt the microtubule structures in the nerve cell, thereby impairing the transport of nutrients as well as the transmission of neuronal messages. It is believed that GSK3 inhibitors may be able to prevent and/or reverse the abnormal hyperphosphorylation of the microtubule-associated protein tau that is an invariant feature of Alzheimer's disease and a number of other neurodegenerative diseases, such as progressive supranuclear palsy, corticobasal degeneration and Pick's disease. Mutations in the tau gene cause inherited forms of fronto-temporal dementia, further underscoring the relevance of tau protein dysfunction for the neurodegenerative process.

The appearances of Paired Helical Filaments, associated with Alzeimer's disease, are caused by the hyperphosphorylation of Tau protein by CDK5-p25. Compounds of the invention are believed to inhibit CDK5 and are therefore believed to be of use in the prevention and treatment of neurodegenerative disorders.

Another embodiment relates to the use of compounds of the invention, or pharmaceutically acceptable salts thereof, in the manufacture of a medicament for treating a metabolic disorder mediated by one or more GSKs. Preferably the medicament is useful in treating metabolic disorders.

Metabolic disorders include Type II diabetes (non insulin dependent diabetes mellitus) and diabetic neuropathy. Compounds of the invention are believed to inhibit GSK-3, which is implicated in Type II diabetes.

GSK3 is one of several protein kinases that phosphorylate glycogen synthase (GS) and is involved in the stimulation of glycogen synthesis by insulin in skeletal muscle. GSK3's action on GS thus results in the latter's deactivation and thus suppression of the conversion of glucose into glycogen in muscles. Type II diabetes (non-insulin dependent diabetes mellitus) is a multi-factorial disease. Hyperglycaemia is due to insulin resistance in the liver, muscles, and other tissues, coupled with impaired secretion of insulin. Skeletal muscle is the main site for insulin-stimulated glucose uptake, there it is either removed from circulation or converted to glycogen. Muscle glycogen deposition is the main determinant in glucose homeostasis and type II diabetics have defective muscle glycogen storage. There is evidence that an increase in GSK3 activity is important in type II diabetes.

Another embodiment relates to the use of compounds of the invention, or pharmaceutically acceptable salts thereof, in the manufacture of a medicament for treating bipolar disorder mediated by one or more kinases. Preferably such medicament is useful in treating bipolar disorder.

Yet another embodiment relates to the use of compounds of the invention, or pharmaceutically acceptable salts thereof, in the manufacture of a medicament for treating a stroke mediated by one or more GSKs. Preferably such medicament is useful in treating a stroke.

Reducing neuronal apoptosis is an important therapeutic goal in the context of head trauma, stroke, epilepsy, and motor neuron disease. GSK3 as a pro-apoptotic factor in neuronal cells makes this protein kinase an attractive therapeutic target for the design of inhibitory drugs to treat these diseases.

Yet another embodiment relates to the use of compounds of the invention, or pharmaceutically acceptable salts thereof, in the manufacture of a medicament for treating alopecia mediated by one or more GSKs. Preferably such medicament is useful in treating alopecia.

The ectopic application of GSK3 inhibitors may be therapeutically useful in the treatment of baldness and in restoring hair growth following chemotherapy-induced alopecia.

A further aspect of the invention relates to a method of treating a condition mediated by one or more enzymes selected from a CDK, aurora kinase, GSK, PLK or tyrosine kinase enzyme as hereinbefore defined.

In one preferred embodiment such condition is a GSK3-dependent disorder, said method comprising administering to a subject in need thereof, a compound of the invention or a pharmaceutically acceptable salt thereof, as defined above in an amount sufficient to inhibit GSK3.

Preferably, the compound of the invention, or pharmaceutically acceptable salt thereof, is administered in an amount sufficient to inhibit GSK3p.

In another preferred embodiment, the invention relates to a method of treating a PLK-dependent disorder, said method comprising administering to a subject in need thereof, a compound of the invention or a pharmaceutically acceptable salt thereof, as defined above in an amount sufficient to inhibit a PLK.

Preferably the compound of the invention is administered in an amount sufficient to inhibit PLK1, PLK2 and/or PLK3.

In another preferred embodiment, the invention relates to a method of treating an aurora kinase-dependent disorder, said method comprising administering to a subject in need thereof, a compound of the invention or a pharmaceutically acceptable salt thereof, as defined above in an amount sufficient to inhibit an aurora kinase.

Preferably the compound of the invention is administered in an amount sufficient to inhibit aurora kinase A, aurora kinase B or aurora kinase C.

In another preferred embodiment, the invention relates to a method of treating a tyrosine kinase-dependent disorder, said method comprising administering to a subject in need thereof, a compound of the invention or a pharmaceutically acceptable salt thereof, as defined above in an amount sufficient to inhibit a tyrosine kinase.

Preferably the compound of the invention is administered in an amount sufficient to inhibit at least one of BCR-ABL, IKK, FLT3, JAK, LCK, PDGF, Src, or VEGF.

In another preferred embodiment, the invention relates to a method of selectively treating a protein kinase-dependent disorder, said method comprising administering to a subject in need thereof, a compound of the invention or a pharmaceutically acceptable salt thereof, as defined above in an amount sufficient to inhibit a selected protein kinase. Preferably said method comprising contacting said protein kinase with a compound of the invention.

Preferably the compound of the invention is administered in an amount sufficient to inhibit at least one of a CDK, GSK, aurora kinase, or PLK, or a tyrosine kinase including, but not limiting to BCR-ABL, IKK, FLT3, JAK, LCK, PDGF, Src, or VEGF.

In a preferred embodiment of this aspect, the protein kinase is a CDK. Preferably, the protein kinase is CDK1, CDK2, CDK3, CDK4, CDK5, CDK6, CDK7, CDK8, CDK9 and CDK11, more preferably CDK2, CDK7 or CDK9.

Known CDK inhibitors under development suffer from a number of problems including a promiscuous kinase inhibitor profile which, apart from multiple CDK inhibition, also potently inhibits other kinases, resulting in observations of toxicity. Other CDK inhibitors under clinical and late-clinical predevelopment are either pan-specific, belonging to the oligo-specific CDK2-CDK7-CDK9 class or are CDK4/6 specific. Although discovery-stage compounds with modest CDK9 selectivity (>10 fold with respect to CDK2 and/or CDK7) have been reported, the determinants for CDK9 selectivity are not currently understood in the published art.

Our research derives from the consideration that apoptotic ability in CLL and other tumour cells can be reinstated by interference with the expression of anti-apoptotic proteins at the transcriptional level via RNAPII, and should provide a therapeutic margin for the elimination of CLL cells while sparing non-transformed quiescent and proliferative cells. Although other CDKs—including CDK1, CDK2, CDK8 and CDK11—have been implicated in the regulation of transcription, the roles of CDK7 and CDK9 appear to be most important in this respect. An important difference between CDK7 and CDK9 is the fact that CDK7 has an additional role as a general CDK-activating kinase (CAK), while CDK9 appears to function exclusively in the regulation of transcription. Apart from regulating transcriptional initiation and elongation, CDK9 also has functions in pre-mRNA splicing.

Results to date strongly suggest that inhibition of CDK9 is necessary and sufficient for effective reversal of apoptotic resistance in CLL. Of all the CDKs involved in RNAPII C-terminal domain (CTD) phosphorylation, CDK9 is unique in apparently lacking cell-cycle related roles. However studies on the effect of depletion of CDK1, CDK2, CDK7 and CDK9 on cellular apoptosis suggest that inhibition of cell cycle CDK functions may not contribute to the elimination of CLL cells and may in fact be undesirable because of antiproliferative effects on nontransformed cells in general, which may manifest as toxicity.

Our research has enabled us to distinguish, both phenotypically and biochemically, between compounds that inhibit RNAP-II CDKs and those that act predominantly through inhibition of cell cycle CDKs (CDK1, CDK2, CDK4, CDK6) or the closely related mitotic kinases.

In one embodiment of the invention the compound of formula I is capable of inhibiting at least one CDK enzyme, preferably at least one of CDK2, CDK7 and CDK9.

Preferably a compound of formula 1 is capable of inhibiting a CDK, more particularly CDK2, CDK7 or CDK9 at sub-micromolar IC₅₀ values, more preferably at IC₅₀ of less than 0.5 micromolar, more preferably less than 0.25 micromolar.

Such compounds of formula I include compounds of formula I′:

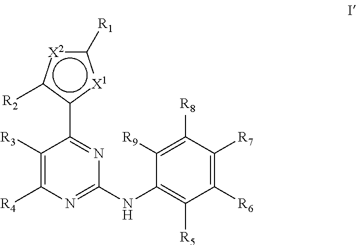

as shown in Table 1 hereinbefore and below, wherein X¹═S, X²═N

| Cpd | R₁ | R₂ | R₃ | R₄ | R₅ | R₆ | R₇ | R₈ | R₉ |
|---|---|---|---|---|---|---|---|---|---|
| 1.1 | NHCH₃ | CH₃ | CN | H | H | “MS” | CH₃ | H | H |
| 1.2 | NHCH₃ | CH₃ | CN | H | H | H | OH | H | H |
| 1.3 | NHCH₃ | CH₃ | CN | H | H | OH | H | H | H |
| 1.4 | NHCH₃ | CH₃ | CN | H | H | “MC” | H | H | H |
| 1.5 | NHCH₃ | CH₃ | CN | H | H | “AcPzC” | H | H | H |
| 1.6 | NHCH₃ | CH₃ | CN | H | H | COOH | H | H | H |
| 1.7 | NHCH₃ | CH₃ | CN | H | H | NO₂ | H | H | H |
| 1.8 | NHCH₃ | CH₃ | CN | H | H | H | SO₂NH₂ | H | H |
| 1.9 | NHCH₃ | CH₃ | CN | H | H | SO₂NH₂ | H | H | H |
| 1.10 | NHCH₃ | CH₃ | CN | H | H | “MePzC” | H | H | H |
| 1.11 | NHCH₃ | CH₃ | CN | H | H | H | “M” | H | H |
| 1.12 | NHCH₃ | CH₃ | CN | H | H | “MS” | H | H | H |
| 1.13 | NHCH₃ | CH₃ | CN | H | H | H | “MS” | H | H |
| 1.14 | NHCH₃ | CH₃ | CN | H | H | SO₂CH₃ | H | H | H |
| 1.15 | NHCH₃ | CH₃ | CN | H | H | “PzC” | H | H | H |
| 1.16 | NH₂ | CH₃ | CN | H | H | “MS” | CH₃ | H | H |
| 1.17 | NH₂ | CH₃ | CN | H | H | H | OH | H | H |
| 1.18 | NH₂ | CH₃ | CN | H | H | OH | H | H | H |
| 1.19 | NH₂ | CH₃ | CN | H | CH₃ | H | OH | CH₃ | H |
| 1.20 | NH₂ | CH₃ | CN | H | H | “MC” | H | H | H |
| 1.21 | NH₂ | CH₃ | CN | H | H | “MePzC” | H | H | H |
| 1.22 | NH₂ | CH₃ | CN | H | H | “AcPzC” | H | H | H |
| 1.23 | NH₂ | CH₃ | CN | H | H | “PzC” | H | H | H |
| 1.24 | NH₂ | H | CN | H | H | “MS” | CH₃ | H | H |
| 1.25 | NH₂ | H | NO₂ | H | H | “MS” | CH₃ | H | H |
| 1.26 | NH₂ | H | CONH₂ | H | H | “MS” | CH₃ | H | H |
| 1.27 | NHCH₃ | CH₃ | CN | H | H | “BPzC” | H | H | H |
| 1.28 | NH₂ | CH₃ | CN | H | H | “BPzC” | H | H | H |
| 1.29 | NH₂ | CH₃ | CN | H | H | “MePdCB” | H | H | H |
| 1.30 | NHCH₃ | CH₃ | CN | H | H | “MePdCB” | H | H | H |
| 1.31 | NH₂ | CH₃ | C(═NH)NH₂ | H | H | “MS” | CH₃ | H | H |
| 1.32 | NH₂ | CH₃ | C(═O)NH₂ | H | H | “MS” | CH₃ | H | H |
| 1.33 | NHCH₃ | CH₃ | C(═O)NH₂ | H | H | “MS” | CH₃ | H | H |
| 1.34 | NHCH₃ | CH₃ | C(═NH)NH₂ | H | H | “MS” | CH₃ | H | H |
| 1.35 | “PyEtA” | CH₃ | C(═NH)NH₂ | H | H | “MS” | CH₃ | H | H |
| 1.36 | “PyEtA” | CH₃ | C(═O)NH₂ | H | H | “MS” | CH₃ | H | H |
| 1.37 | “PyMeA” | CH₃ | CN | H | H | “MePzC” | H | H | H |
| 1.38 | “PyMeA” | CH₃ | CN | H | H | “PzC” | H | H | H |
| 1.39 | NH(CH₂)₂CH₃ | CH₃ | CN | H | H | “PzC” | H | H | H |
| 1.40 | NHCH₃ | CH₃ | CN | H | H | (2-hydroxyethyl)“PzC” | H | H | H |
| 1.41 | NHCH₃ | CH₃ | CN | H | H | (2-methoxyethyl)“PzC” | H | H | H |
| 1.42 | NH₂ | CH₃ | CN | H | H | (2-methoxyethyl)“PzC” | H | H | H |
| 1.43 | NH(CH₂)₂CH₃ | CH₃ | CN | H | H | (2-methoxyethyl)“PzC” | H | H | H |
| 1.44 | NH(CH₂)₂CH₃ | CH₃ | CN | H | H | (2-methoxyethyl)“PdC” | H | H | H |
| 1.45 | NH(CH₂)₂CH₃ | CH₃ | CN | H | H | H | “MeDz” | H | H |
| 1.46 | NHCH₂CH₃ | CH₃ | CN | H | H | H | “MeDz” | H | H |
| 1.47 | NHCH₃ | CH₃ | CN | H | H | H | “MeDz” | H | H |
| 1.48 | NH₂ | CH₃ | CN | H | H | H | “MeDz” | H | H |
| 1.49 | NH₂ | CH₃ | CN | H | H | “MeDz” | H | H | H |
| 1.50 | NHCH₃ | CH₃ | CN | H | H | “MeDz” | H | H | H |
| 1.51 | NHCH₂CH₃ | CH₃ | CN | H | H | “MeDz” | H | H | H |
| 1.52 | NH(CH₂)₂CH₃ | CH₃ | CN | H | H | “MeDz” | H | H | H |
| 1.53 | NHCH₃ | CH₃ | CN | H | CH₃ | H | OH | CH₃ | H |

and Table 2 wherein: X¹═N, X²═S

| Cpd | R₁ | R₂ | R₃ | R₄ | R₅ | R₆ | R₇ | R₈ | R₉ |
|---|---|---|---|---|---|---|---|---|---|
| 1.54 | NHCH₃ | H | CN | H | H | “MS” | CH₃ | H | H |
| 1.55 | NHCH₃ | H | NO₂ | H | H | “MS” | CH₃ | H | H |
| 1.56 | NHCH₃ | H | C(═O)NH₂ | H | H | “MS” | CH₃ | H | H |

and compounds of formula I″:

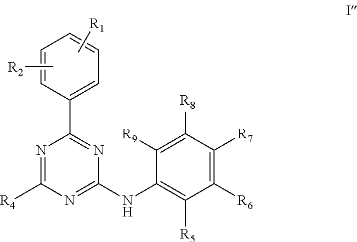

as shown in Table 3

| cpd | R₁ | R₂ | R₄ | R₅ | R₆ | R₇ | R₈ | R₉ |
|---|---|---|---|---|---|---|---|---|
| 2.0 | 3-OCH₃ | H | Cl | H | H | OH | H | H |
| 2.1 | 3-OCH₃ | H | Cl | H | H | OH | H | H |
| 2.2 | 3-OCH₃ | H | Cl | H | NO₂ | H | H | H |
| 2.3 | 3-OCH₃ | H | Cl | H | Br | H | H | H |
| 2.4 | 3-OCH₃ | H | Cl | H | “MS” | CH₃ | H | H |
| 2.5 | 3-OCH₃ | H | NH₂ | H | H | OH | H | H |
| 2.6 | 3-OCH₃ | H | NH₂ | H | NO₂ | H | H | H |
| 2.7 | 3-OCH₃ | H | NHCH₃ | H | NO₂ | H | H | H |
| 2.8 | 3-OCH₃ | H | NHOH | H | NO₂ | H | H | H |
| 2.9 | 3-OCH₃ | H | NH₂ | H | Br | H | H | H |
| 2.10 | 3-OCH₃ | H | NH₂ | H | “MS” | CH₃ | H | H |
| 2.11 | 3-OCH₃ | H | NH₂ | H | “MC” | H | H | H |
| 2.12 | 3-OCH₃ | H | NH₂ | H | “PzC” | H | H | H |

and compounds of formula I′:

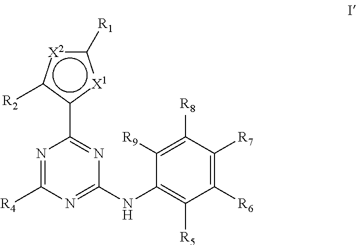

as shown in Table 4 wherein X¹═S, X²═N

| cpd | R₁ | R₂ | R₄ | R₅ | R₆ | R₇ | R₈ | R₉ |
|---|---|---|---|---|---|---|---|---|
| 3.2 | NMeBoc | Ph | Cl | H | NO₂ | H | H | H |
| 3.3 | NMeBoc | Ph | NH₂ | H | NO₂ | H | H | H |
| 3.4 | NHMe | Ph | NH₂ | H | NO₂ | H | H | H |
| 3.5 | NHCH₃ | Ph | NH₂ | H | “MS” | CH₃ | H | H |
| 3.6 | NHCH₃ | tBu | NH₂ | H | “MS” | CH₃ | H | H |
| 3.7 | NHCH₃ | “Py” | NH₂ | H | “MS” | CH₃ | H | H |
| 3.8 | NHCH₃ | CH₃ | NH₂ | H | “MS” | CH₃ | H | H |
| 3.9 | NHCH₃ | CH₃ | NH₂ | H | “MS” | H | H | H |
| 3.10 | NH₂ | CH₃ | NH₂ | H | “MS” | H | H | H |
| 3.11 | NH₂ | Ph | NH₂ | H | “MS” | H | H | H |
| 3.12 | NH₂ | Ph | NH₂ | H | “MS” | CH₃ | H | H |
| 3.13 | NH₂ | Ph | NH₂ | H | “PzS” | H | H | H |
| 3.14 | NH₂ | Ph | NH₂ | H | “MePzS” | H | H | H |
| 3.15 | NH₂ | CH₃ | NH₂ | H | “BPzS” | H | H | H |
| 3.16 | NHCH₃ | CH₃ | NH₂ | H | “BPzS” | H | H | H |
| 3.17 | NHCH₂CH₃ | CH₃ | NH₂ | H | “BPzS” | H | H | H |
| 3.18 | NHCH₂CH₃ | CH₃ | NH₂ | H | “MePzS” | H | H | H |
| 3.19 | NHCH₃ | CH₃ | NH₂ | H | “MePzS” | H | H | H |
| 3.20 | NHCH₃ | CH₃ | NH₂ | H | “PzS” | H | H | H |
| 3.21 | NH₂ | CH₃ | NH₂ | H | “PzS” | H | H | H |
| 3.22 | NH₂ | CH₃ | NH₂ | H | “PzC” | H | H | H |
| 3.23 | NH₂ | CH₃ | NH₂ | H | “MePzC” | H | H | H |
| 3.24 | NHCH₃ | CH₃ | NH₂ | H | “MePzC” | H | H | H |
| 3.25 | NHCH₂CH₃ | CH₃ | NH₂ | H | “MePzC” | H | H | H |
| 3.26 | NHCH₃ | CH₃ | NH₂ | H | “PdC” | H | H | H |
| 3.27 | NHCH₃ | CH₃ | NH₂ | H | “MePdC” | H | H | H |
| 3.28 | NHCH₃ | CH₃ | NH₂ | H | “BPdC” | H | H | H |
| 3.29 | NH₂ | CH₃ | NH₂ | H | “Pz” | H | H | H |
| 3.30 | NH₂ | CH₃ | NH₂ | H | “MePz” | H | H | H |

wherein abbreviated substituents in the above Tables are as given hereinabove.

In a further preferred embodiment compounds of formula I are capable of exhibiting an antiproliferative effect in human cell lines, as measured by a standard 72h MTT cytotoxicity assay. Preferably the compound of formula I exhibits an IC₅₀ value of less than 1 micromolar.

Such compounds of formula I include compounds of formula I′:

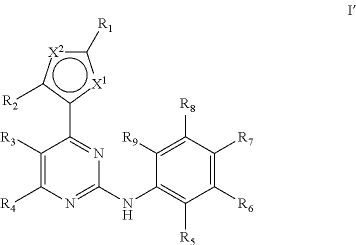

as shown in Table 1 hereinabove and below wherein X¹═S, X²═N

| Cpd | R₁ | R₂ | R₃ | R₄ | R₅ | R₆ | R₇ | R₈ | R₉ |
|---|---|---|---|---|---|---|---|---|---|
| 1.1 | NHCH₃ | CH₃ | CN | H | H | “MS” | CH₃ | H | H |
| 1.2 | NHCH₃ | CH₃ | CN | H | H | H | OH | H | H |
| 1.3 | NHCH₃ | CH₃ | CN | H | H | OH | H | H | H |
| 1.4 | NHCH₃ | CH₃ | CN | H | H | “MC” | H | H | H |
| 1.5 | NHCH₃ | CH₃ | CN | H | H | “AcPzC” | H | H | H |
| 1.6 | NHCH₃ | CH₃ | CN | H | H | COOH | H | H | H |
| 1.7 | NHCH₃ | CH₃ | CN | H | H | NO₂ | H | H | H |
| 1.8 | NHCH₃ | CH₃ | CN | H | H | H | SO₂NH₂ | H | H |
| 1.9 | NHCH₃ | CH₃ | CN | H | H | SO₂NH₂ | H | H | H |
| 1.10 | NHCH₃ | CH₃ | CN | H | H | “MePzC” | H | H | H |
| 1.11 | NHCH₃ | CH₃ | CN | H | H | H | “M” | H | H |
| 1.12 | NHCH₃ | CH₃ | CN | H | H | “MS” | H | H | H |
| 1.13 | NHCH₃ | CH₃ | CN | H | H | H | “MS” | H | H |
| 1.14 | NHCH₃ | CH₃ | CN | H | H | SO₂CH₃ | H | H | H |
| 1.15 | NHCH₃ | CH₃ | CN | H | H | “PzC” | H | H | H |
| 1.16 | NH₂ | CH₃ | CN | H | H | “MS” | CH₃ | H | H |
| 1.17 | NH₂ | CH₃ | CN | H | H | H | OH | H | H |
| 1.18 | NH₂ | CH₃ | CN | H | H | OH | H | H | H |
| 1.19 | NH₂ | CH₃ | CN | H | CH₃ | H | OH | CH₃ | H |
| 1.20 | NH₂ | CH₃ | CN | H | H | “MC” | H | H | H |
| 1.21 | NH₂ | CH₃ | CN | H | H | “MePzC” | H | H | H |
| 1.22 | NH₂ | CH₃ | CN | H | H | “AcPzC” | H | H | H |
| 1.23 | NH₂ | CH₃ | CN | H | H | “PzC” | H | H | H |
| 1.24 | NH₂ | H | CN | H | H | “MS” | CH₃ | H | H |
| 1.25 | NH₂ | H | NO₂ | H | H | “MS” | CH₃ | H | H |
| 1.26 | NH₂ | H | CONH₂ | H | H | “MS” | CH₃ | H | H |
| 1.27 | NHCH₃ | CH₃ | CN | H | H | “BPzC” | H | H | H |
| 1.28 | NH₂ | CH₃ | CN | H | H | “BPzC” | H | H | H |
| 1.29 | NH₂ | CH₃ | CN | H | H | “MePdCB” | H | H | H |
| 1.30 | NHCH₃ | CH₃ | CN | H | H | “MePdCB” | H | H | H |
| 1.31 | NH₂ | CH₃ | C(═NH)NH₂ | H | H | “MS” | CH₃ | H | H |
| 1.32 | NH₂ | CH₃ | C(═O)NH₂ | H | H | “MS” | CH₃ | H | H |
| 1.33 | NHCH₃ | CH₃ | C(═O)NH₂ | H | H | “MS” | CH₃ | H | H |
| 1.34 | NHCH₃ | CH₃ | C(═NH)NH₂ | H | H | “MS” | CH₃ | H | H |
| 1.35 | “PyEtA” | CH₃ | C(═NH)NH₂ | H | H | “MS” | CH₃ | H | H |
| 1.36 | “PyEtA” | CH₃ | C(═O)NH₂ | H | H | “MS” | CH₃ | H | H |
| 1.37 | “PyMeA” | CH₃ | CN | H | H | “MePzC” | H | H | H |
| 1.38 | “PyMeA” | CH₃ | CN | H | H | “PzC” | H | H | H |
| 1.39 | NH(CH₂)₂CH₃ | CH₃ | CN | H | H | “PzC” | H | H | H |
| 1.40 | NHCH₃ | CH₃ | CN | H | H | (2-hydroxyethyl)“PzC” | H | H | H |
| 1.41 | NHCH₃ | CH₃ | CN | H | H | (2-methoxyethyl)“PzC” | H | H | H |
| 1.42 | NH₂ | CH₃ | CN | H | H | (2-methoxyethyl)“PzC” | H | H | H |
| 1.43 | NH(CH₂)₂CH₃ | CH₃ | CN | H | H | (2-methoxyethyl)“PzC” | H | H | H |
| 1.44 | NH(CH₂)₂CH₃ | CH₃ | CN | H | H | (2-methoxyethyl)“PdC” | H | H | H |
| 1.45 | NH(CH₂)₂CH₃ | CH₃ | CN | H | H | H | “MeDz” | H | H |
| 1.46 | NHCH₂CH₃ | CH₃ | CN | H | H | H | “MeDz” | H | H |
| 1.47 | NHCH₃ | CH₃ | CN | H | H | H | “MeDz” | H | H |
| 1.48 | NH₂ | CH₃ | CN | H | H | H | “MeDz” | H | H |
| 1.49 | NH₂ | CH₃ | CN | H | H | “MeDz” | H | H | H |
| 1.50 | NHCH₃ | CH₃ | CN | H | H | “MeDz” | H | H | H |
| 1.51 | NHCH₂CH₃ | CH₃ | CN | H | H | “MeDz” | H | H | H |
| 1.52 | NH(CH₂)₂CH₃ | CH₃ | CN | H | H | “MeDz” | H | H | H |
| 1.53 | NHCH₃ | CH₃ | CN | H | CH₃ | H | OH | CH₃ | H |

and Table 2 wherein: X¹═N, X²═S

| Cpd | R₁ | R₂ | R₃ | R₄ | R₅ | R₆ | R₇ | R₈ | R₉ |
|---|---|---|---|---|---|---|---|---|---|
| 1.54 | NHCH₃ | H | CN | H | H | “MS” | CH₃ | H | H |
| 1.55 | NHCH₃ | H | NO₂ | H | H | “MS” | CH₃ | H | H |
| 1.56 | NHCH₃ | H | C(═O)NH₂ | H | H | “MS” | CH₃ | H | H |

and compounds of formula I″:

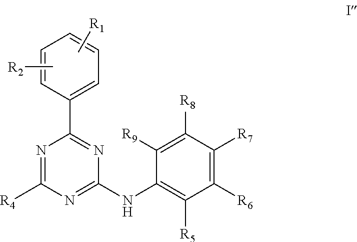

as shown in Table 3a:

| cpd | R₁ | R₂ | R₄ | R₅ | R₆ | R₇ | R₈ | R₉ |
|---|---|---|---|---|---|---|---|---|
| 2.0 | 3-OCH₃ | H | Cl | H | H | OH | H | H |
| 2.4 | 3-OCH₃ | H | Cl | H | “MS” | CH₃ | H | H |

wherein abbreviated substituents in the above Tables are as given hereinabove.

In a further aspect of the invention there is provided a method of treating a proliferative disease or disorder, a viral disorder, a cardiovascular disease, a CNS disorder, an autoimmune disease, a metabolic disorder, stroke, alopecia, an inflammatory disease or an infectious disease, said method comprising administering to a subject in need thereof, a compound of formula I as hereinbefore defined in an effective amount.

The use of a compound of the invention in the manufacture of a medicament as hereinbefore defined includes the use of the compound directly, or in any stage of the manufacture of such a medicament, or in vitro in a screening programme to identify further agents for the prevention or treatment of the hereinbefore defined diseases or conditions.

A further aspect of the invention relates to the use of a compound of formula I or a pharmaceutically acceptable salt or solvate or physiologically hydrolysable, solubilising or immobilising derivative thereof, in an assay for identifying candidate compounds capable of treating one or more disorders or diseases as hereinbefore defined. Preferably a compound is of use in identifying candidate compounds capable of inhibiting a protein kinase, more preferably one or more of a CDK, aurora kinase, GSK, PLK or tyrosine kinase enzyme.

Pharmaceutical Compositions

In a further aspect of the invention there is provided a pharmaceutical composition comprising a therapeutically effective amount of a compound of formula I or its physiologically acceptable salt and physiologically hydrolysable derivative as hereinbefore defined in association with one or more pharmaceutical carriers, excipients or diluents. Suitable carriers, excipients or diluents may be selected having regard to the intended mode of administration and standard practice. The pharmaceutical compositions may be for human or animal usage in human and veterinary medicine, preferably for treatment of a condition, disease or disorder as hereinbefore defined or in inhibiting one or more protein kinase enzyme, more preferably one or more of a CDK, aurora kinase, GSK, PLK or tyrosine kinase enzyme.

Examples of suitable carriers include lactose, starch, glucose, methyl cellulose, magnesium stearate, mannitol, sorbitol and the like.

A therapeutically effective amount is any amount from 0.1% to 99.9% w/w.

A composition of the invention is suitably for any desired mode of administration including oral, rectal, vaginal, parenteral, intramuscular, intraperitoneal, intraarterial, intrathecal, intrabronchial, subcutaneous, intradermal, intravenous, nasal, buccal or sublingual and the like.

A composition for oral administration is suitably formulated as a compressed tablet, tablet, capsule, gel capsule, powder, solution, dispersion, suspension, drops or the like. Such forms may be produced according to known methods and may include any suitable binder, lubricant, suspending agent, coating agent or solubilising agent or combinations thereof.

A composition for administration by means of injection is suitably formulated as a sterile solution or emulsion from a suitable solution or powder. Alternatively a composition may be in the form of suppositories, pessaries, suspensions, emulsions, lotions, creams, ointments, skin patches, gels, solgels, sprays, solutions or dusting powders.

An indicated daily dosage is from about 1 mg to about 1000 mg and compositions generally contain from about 0.25 mg to about 250 mg of the active ingredient per dose.

A composition may include one or more additional active ingredients or may be administered together with compositions comprising other active ingredients for the treatment of the same, or different condition. Coadministration may be simultaneously, consecutively or sequentially.

An additional active ingredient is suitably selected from other existing anticancer agents. This may be desirable to prevent an overlap of major toxicities, mechanism of action and resistance mechanisms and to enable administration of drugs at their maximum tolerated doses with minimum time intervals between doses. Coadministration is also favoured to promote additive or possible synergistic effects. Selection of other active ingredients and regime of administration may be having regard to a knowledge of agents which are effective in treatment of cell lines derived from the cancer to be treated.

Suitable anti-proliferative agents that may be used in combination with a compound of the invention include DNA damaging agents, anti-metabolites, anti-tumour antibiotics, dihydrofolate reductase inhibitors, pyrimidine analogues, purine analogues, cyclin-dependant kinase inhibitors, thymidylate synthase inhibitors, DNA intercalators, DNA cleavers, topoisomerase inhibitors, anthracyclines, vinca drugs, mitomycins, bleomycins, cytotoxic nucleosides, pteridine drugs, diynenes, podophyllotoxins, platinum containing drugs, differentiation inducers and taxanes. Suitable examples of these drugs are known in the art.

In a particular advantage the compounds of the invention display a CDK and cell line selectivity which is not displayed by known anti-proliferative drugs and therefore co-administration is recommended having regard to desired selectivity.

A compound as hereinbefore defined may be in free form, i.e. normally as a base, or in any suitable salt or ester form. Free forms of the compound may be converted into salt or ester form and vice versa, in conventional manner. Suitable salts include hydrochloride, dihydrochloride, hydroformate, amide, succinate, half succinate, maleate, acetate, trifluoroacetate, fumarate, phthalate, tetraphthalate, benzoate, sulfonate, sulphate, phosphate, oxalate, malonate, hydrogen malonate, ascorbate, glycolate, lactate, malate, tartarate, citrate, aspartate or glutamate and variants thereof. Suitable acids for acid addition salt formation include the corresponding acids, i.e. hydrochloric, formic, amino acid, succinic, maleic, acetic, trifluoroacetic, fumaric, phthalic, tetraphthalic, benzoic, sulfonic, sulphuric, phosphoric, oxalic, malonic, ascorbic, glycolic, lactic, malic, tartaric, citric, aspartic or glutamic acids and the like.

Suitable esters include those obtained with the above acids, with hydroxides such as sodium, potassium, calcium or the like, or with alcohols.

The compounds of formula I may be present as one or both enantiomeric or tautomeric forms, or stereo or geometric isomeric forms, where relevant. Such forms may be identified and prepared or isolated by methods known in the art. Reference herein to compounds of formula I also encompasses reference to crystalline forms, polymorphs, hydrous and anhydrous forms and prodrugs thereof.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention-is-not; restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### EXAMPLES

Synthesis of Compounds

General. ¹H-NMR spectra were obtained using a Bruker-400 spectrometer. Chemical shifts are reported in parts per million relative to internal tetramethylsilane standard. Coupling constants (J) are quoted to the nearest 0.1 Hz. The following abbreviations are used: s, singlet; d, doublet; t, triplet; q, quartet; qu, quintuplet; m, muiliplet and br, broad. Mass spectra were obtained using a Waters 2795 single quadrupole mass spectrometer with electrospray ionization (ESI). Microwave assisted chemistry was carried out using CEM Discovery model (Biotage Ltd. UK). TLC (thin-layer chromatography) was performed using alumina plates coated with silica gel G60. Developed plates were air dried and analysed under a UV lamp (254/365 nm). Silica gel (EM Kieselgel 60, 0.040-0.063 mm, Merck) or ISOLUTE pre-packed columns was used for flash chromatography. Melting points (mp) were determined with an Electrothermal melting point apparatus and are uncorrected.

### Example 1

### Preparation of a Compound of Formula I

### 1.1 4-(4-Methyl-2-methylamino-thiazol-5-yl)-2-[4-methyl-3-(morpholine-4-sulfonyl)-phenylamino]-pyrimidine-5-carbonitrile

To a solution of 1-(4-methyl-2-methylamino-thiazol-5-yl)ethanone (14.5 mmol) in 3 mL acetic acid and 10 ml dichloromethane cooling on an ice bath bromine (14.5 mmol) was added dropwise. The reaction mixture was stirred for 1.5 hours. If the reaction mixture turns to cake further 3 mL of AcOH can be added. The solvent was evaporated in vacuo. The residue was partitioned between CH₂Cl₂ and saturated aq. NaHCO₃. The organic layer was washed with brine, dried over Na₂SO₄, and evaporated to give the crude 2-bromo-1-(4-methyl-2-methylamino-thiazol-5-yl)-ethanone. Buff cream solid (95% yield): mp 149-150° C. ¹H-NMR (DMSO-d₆) δ: 2.35 (s, 3H, CH₃), 2.45 (s, 3H, CH₃), 2.85 (s, 3H, CH₃), 8.48 (s, 1H, NH). HRMS (ESI) 171.0577 (M+H)⁺.

To a solution of 2-bromo-1-(4-methyl-2-methylamino-thiazol-5-yl)-ethanone (10 mmol) in ethanol (8 mL) was added a solution of NaCN (20 mmol) in 4 mL H₂O. The reaction mixture was stirred at r.t. for 1 hour. The mixture was filled and the filtrate was concentrated in vacuo. The residue was poured into 30 mL ice water and stirred for 3 h. The precipitates were collected and dried to give 3-(4-methyl-2-methylamino-thiazol-5-yl)-3-oxo-propionitrile. Light yellow solid: ¹H-NMR (DMSO-d₆) δ: 2.45 (s, 3H, CH₃), 2.86 (s, 3H, CH₃), 4.38 (s, 2H, CH₂), 8.65 (s, 1H, NH). HRMS (ESI) 194.0365 (M−H)⁻.

3-(4-Methyl-2-methylamino-thiazol-5-yl)-3-oxo-proplonitrile (8 mmol) was treated with 24 mmol of N,N-dimethylformamide dimethylacetal in reflux for 3 hrs. The reaction mixture was concentrated in vacuo and purified by column chromatography to give 3-dimethylamino-2-(4-methyl-2-methylamino-thiazole-5-carbonyl)-acrylonitrile. Orange solid: ¹H-NMR (DMSO-d₆) δ 2.33 (s, 3H, CH₃), 2.82 (d, J=4.8 Hz, 3H, CH₃), 3.26 (s, 3H, CH₃), 3.32 (s, 3H, CH₃), 7.80 (s, 1H, CH), 8.09 (t, J=4.8 Hz, 1H, NH). HRMS (ESI) 250.9323 (M+H)⁺.

A mixture of 3-dimethylamino-2-(4-methyl-2-methylamino-thiazole-5-carbonyl)-acrylonitrile and an equimolar amount of N-[4-methyl-3-(morpholine-4-sulfonyl)-phenyl]-guanidine in 2-methoxyethanol was microwaved at 140° C. for 30 min. The mixture was evaporate in vacuo and purified by column chromatography using EtOAC to elute the desired 4-(4-methyl-2-methylamino-thiazol-5-yl)-2-[4-methyl-3-(morpholine-4-sulfonyl)-phenylamino]-pyrimidine-5-carbonitrile. Yellow solid. m.p. 245-246° C. ¹H-NMR (DMSO-d₆) δ: 2.46 (s, 3H, CH₃), 2.89 (d, 3H, J=4.4 Hz, CH₃), 3.05 (t, 4H, J=4.4 Hz, CH₂×2), 3.63 (t, 4H, J=4.4 Hz, CH₂×2), 7.43 (d, 1H, J=8.4 Hz, Ph-H), 7.95 (dd, 1H, J=8.4, 1.6 Hz, Ph-H), 8.18 (d, 1H, J=2.0 Hz, Ph-H), 8.29 (q, 1H, J=4.8 Hz, NH), 8.82 (s, 1H, Pyimidinyl-H), 10.46 (bs, 1H, NH). HRMS (ESI) 486.1421 (M+H⁺. C₂₁H₂₃N₇O₃S₂ requires 486.1304).

The following compounds were synthesised by an analogous route:

### 1.2 2-(4-Hydroxy-phenylamino)-4-(4-methyl-2-methylamino-thiazol-5-yl)-pyrimidine-5-carbonitrile

Prepared by treatment of 3-dimethylamino-2-(4-methyl-2-methylamino-thiazole-5-carbonyl)-acrylonitrile and N-(4-hydroxy-phenyl)-guanidine hydrochloride. HRMS (ESI) 339.1089 (M+H⁺. C₁₆H₁₄N₆OS requires 339.0950).

### 1.3 2-(3-Hydroxy-phenylamino)-4-(4-methyl-2-methylamino-thiazol-5-yl)-pyrimidine-5-carbonitrile

Prepared by treatment of 3-dimethylamino-2-(4-methyl-2-methylamino-thiazole-5-carbonyl)-acrylonitrile and N-(3-hydroxy-phenyl)-guanidine hydrochloride. HRMS (ESI) 339.1078 (M+H⁺. C₁₆H₁₄N₆OS requires 339.0950).

### 1.4 4-(4-Methyl-2-methylamino-thiazol-5-yl)-2-[3-(morpholine-4-carbonyl)-phenylaminol-pyrimidine-5-carbonitrile

Prepared by treatment of 3-dimethylamino-2-(4-methyl-2-methylamino-thiazole-5-carbonyl)-acrylonitrile and N-[3-(morpholine-4-carbonyl)-phenyl]-guanidine hydrochloride. HRMS (ESI) 436.1616 (M+H⁺. C₂₁H₂₁N₇O₂S requires 436.1477).

### 1.5 2-(3-(4-Acetylpiperazine-1-carbonyl)phenylamino)-4-(4-methyl-2-(methylamino)-thiazol-5-yl)pyrimidine-5-carbonitrile

Prepared by treatment of 3-dimethylamino-2-(4-methyl-2-methylamino-thiazole-5-carbonyl)-acrylonitrile and 1-(3-(4-acetyl-piperazine-1-carbonyl)phenyl)guanidine. Yellow solid. HRMS (ESI) 477.1973 (M+H⁺. C₂₃H₂₄N₈O₂S requires 477.1743).

### 1.6 3-(5-cyano-4-(4-methyl-2-(methylamino)thiazol-5-yl)pyrimidin-2-ylamino)benzoic acid

Prepared from 2-(3-(4-acetylpiperazine-1-carbonyl)phenylamino)-4-(4-methyl-2-(methylamino)-thiazol-5-yl)pyrimidine-5-carbonitrile. Yellow solid HRMS (ESI) 367.1093 (M+H⁺. C₁₇H₁₄N₆O₂S requires 367.0899).

### 1.7 4-(4-methyl-2-(methylamino)thiazol-5-yl)-2-(3-nitrophenylamino)pyrimidine-5-carbonitrile

Prepared by treatment of 3-dimethylamino-2-(4-methyl-2-methylamino-thiazole-5-carbonyl)-acrylonitrile and 1-(3-nitrophenyl)guanidine hydrochloride. HRMS (ESI) 366.0768 (M−H⁻. C₁₆H₁₃N₇O₂S requires 366.0851).

### 1.8 4-(5-cyano-4-(4-methyl-2-(methylamino)thiazol-5-yl)pyrimidin-2-ylamino)benzenesulfonamide

Prepared by treatment of 3-dimethylamino-2-(4-methyl-2-methylamino-thiazole-5-carbonyl)-acrylonitrile and 4-guanidinobenzenesulfonamide. HRMS (ESI) 400.0634 (M−H⁻. C₁₆H₁₅N₇O₂S₂ requires 400.0729).

### 1.9 3-(5-cyano-4-(4-methyl-2-(methylamino)thiazol-5-yl)pyrimidin-2-ylamino)benzenesulfonamide

Prepared by treatment of 3-dimethylamino-2-(4-methyl-2-methylamino-thiazole-5-carbonyl)-acrylonitrile and 3-guanidinobenzenesulfonamide. HRMS (ESI) 401.8300 (M+H⁺. C₁₆H₁₅N₇O₂S₂ requires 401.0729).

### 1.10 4-(4-methyl-2-(methylamino)thiazol-5-yl)-2-(3-(4-methylpiperazine-1-carbonyl)phenylamino)pyrimidine-5-carbonitrile

Prepared by treatment of 3-dimethylamino-2-(4-methyl-2-methylamino-thiazole-5-carbonyl)-acrylonitrile and 1-(3-(4-methylpiperazine-1-carbonyl)phenyl)guanidine. Yellow solid. HRMS (ESI) 448.8561 (M+H⁺. C₂₂H₂₄N₈OS requires 448.1794).

### 1.11 4-(4-methyl-2-(methylamino)thiazol-5-yl)-2-(4-morpholinophenylamino)pyrimidine-5-carbonitrile

Prepared by treatment of 3-dimethylamino-2-(4-methyl-2-methylamino-thiazole-5-carbonyl)-acrylonitrile and 1-(4-morpholinophenyl)guanidine. Yellow solid. HRMS (ESI) 407.8810 (M+H⁺. C₂₀H₂₁N₇OS requires 407.1528).

### 1.12 4-(4-methyl-2-(methylamino)thiazol-5-yl)-2-(3-(morpholinosulfonyl)phenyl-amino)pyrimidine-5-carbonitrile

Prepared by treatment of 3-dimethylamino-2-(4-methyl-2-methylamino-thiazole-5-carbonyl)-acrylonitrile and 1 -(3-(morpholinosulfonyl)phenyl)guanidine. Yellow solid. HRMS (ESI) 471.7335 (M+H⁺. C₂₀H₂₁N₇O₃S₂ requires 471.1147.

### 1.13 4-(4-methyl-2-(methylamino)thiazol-5-yl)-2-(4-(morpholinosulfonyl)phenyl-amino)pyrimidine-5-carbonitrile

Prepared by treatment of 3-dimethylamino-2-(4-methyl-2-methylamino-thiazole-5-carbonyl)-acrylonitrile and 1-(4-(morpholinosulfonyl)phenyl)guanidine. Yellow solid. HRMS (ESI) 471.7248 (M+H⁺. C₂₀H₂₁N₇O₃S₂ requires 471.1147.

### 1.14 4-(4-methyl-2-(methylamino)thiazol-5-yl)-2-(3-(methylsulfonyl)phenylamino)pyrimidine-5-carbonitrile

Prepared by treatment of 3-dimethylamino-2-(4-methyl-2-methylamino-thiazole-5-carbonyl)-acrylonitrile and 1-(3-(methylsulfonyl)phenyl)guanidine. Yellow solid. HRMS (ESI) 400.8207 (M+H⁺. C₁₇H₁₆N₆O₂S₂ requires 400.0776.

### 2.0 4-[4-Chloro-6-(3-methoxy-phenyl)-[1,3,5]triazin-2-ylamino]-phenol

A solution of 2,4,6-trichloro-[1,3,5]-triazine (20 mmol) in toluene cooling on an ice bath was treated with 3-methoxphenyl magnesium bromide (20 mmol) dropwise. The reaction mixture was stirred for 2 h and warm to room temperature. The compound was evaporated to dryness in vacuo to give 2,4-dichloro-6-(3-methoxy-phenyl)-[1,3,5]triazine as white solid. MS (ESI⁺) m/z 256.00 (M+H)⁺.

Above compound in MeCN was treated with 4-aminophenol (1 equiv molar) in the presence of diisopropylamine at room temperature for 2 hrs. The reaction mixture was purified by flash chromatography using EtOAc-PE (2:1, v/v) to elute 4-[4-chloro-6-(3-methoxy-phenyl)-[1,3,5]triazin-2-ylamino]-phenol as a yellow solid. MS (ESI⁺) m/z 328.06

### 2.1 4-(4-chloro-6-(3-methoxyphenyl)-1,3,5-triazin-2-ylamino)phenol

A small crystal of I₂ was placed in an oven-dried flask containing Mg (1.07 g, 44.58 mmol, 1.8 eq.) in dry THF (15 ml) to activate the metal. After the coloration disappeared, a solution of 3-bromoanisole (4.64 g, 24.81 mmol, 1 eq.) in dry THF (15 ml) was added dropwise keeping the reaction mixture warm. After the addition completed, the reaction was stirred for 30 min (the progress of the reaction was monitored by TLC).

Cyanuric chloride (4.58 g, 24.81 mmol, 1 eq.) was dissolved in 20 ml of dry THF in an oven-dried three-necked flask equipped with a dropping funnel, a bubbler and a thermometer. The resulting solution was cooled down to −20° C. and the Grignard reagent was added dropwise via the dropping funnel keeping temperature of the reaction mixture below −15° C. during the addition. After the addition completed, the content of the flask was stirred for 45 min at −15° C. The reaction mixture was quenched with water (100 ml), extracted with ethyl acetate (3×50 ml). Combined organic extracts were washed with brine (50 ml), dried over MgSO₄. The solvent was evaporated under reduced pressure and the residue was subjected to a flash column chromatography eluting with a mixture of petroleum ether (40-60° C.) diethyl ether=90:10 to yield 2.65 g (42%) of 2,4-dichloro-6-(3′-methoxyphenyl)-1,3,5-triazine as a white solid, mp 113.8-114.0° C. (from petroleum ether (40-60° C.)); δ_{H} (400 MHz, CDCl₃) 8.11 (1H, ddd, J 7.8, 1.4 and 1.0, 6′-H), 7.99 (1H, dd, J 2.7 and 1.4, 2′-H), 7.43 (1H, app. t, J 8.0, 5′-H), 7.19 (1H, ddd, 8.2, 2.7 and 1.0, 4′-H), 3.91 (3H, s, OCH₃)

2,4-Dichloro-6-(3′-methoxyphenyl)-1,3,5-triazine (0.379 g, 1.48 mmol, 1 eq.) was dissolved in DMF (7 ml) in the presence of NaHCO₃ (0.249 g, 2.96 mmol, 2 eq.) and 4-aminophenol (1.48 mmol, 1 eq.) was introduced into the flask. The reaction mixture was allowed to stir at room temperature till the starting materials disappeared by TLC. The reaction mixture was quenched with water (30 ml). Resulted precipitate was filtered and washed with several times with water. The aqueous solution was extracted with ethyl acetate (3×10 ml). Combined organic layers were washed with brine (10 ml). The combined organic solution was dried over MgSO₄. The solvent was evaporated under reduced pressure and the residue was subjected to a flash column chromatography to yield 0.481 g (99%) of yellow solid eluting with petroleum ether (40-60° C.):EtOAc=70:30; mp 164.1-164.3° C. (from toluene); δ_{H} (400 MHz, DMSO-d₆) 10.50 and 1046 (1H, 2×s), 9.38 and 9.38 (1H, 2×s), 7.79-7.93 (2H, m, Ar), 7.39-7.51 (3H, m, Ar), 7.19-7.21 (1H, m, Ar), 6.76-6.81 (2H, m, Ar), 3.83 and 3.82 (3H, 2×s, OCH₃); HRMS (ESI) 329.0823 (M+H⁺. C₁₆H₁₄N₄³⁵ClO₂ requires 329.0805).

The following compounds were synthesised by an analogous route.

### 2.2 4-chloro-6-(3-methoxyphenyl)-N-(3-nitrophenyl)-1,3,5-triazin-2-amine

Prepared using 2,4-dichloro-6-(3′-methoxyphenyl)-1,3,5-triazine (0.379 g, 1.48 mmol), NaHCO₃ (0.249 g, 2.96 mmol) and 3-nitroaniline (0.204 g, 1.48 mmol) to yield 0.518 g (98%) of a yellow solid eluting with petroleum ether (40-60° C.):AcOEt=80:20; mp 154.7-154.9° C.; HRMS (ESI) 358.0749 (M+H⁺. C₁₆H₁₃N₅O₃ ³⁵Cl requires 358.0707).

### 2.3 N-(3-bromophenyl)-4-chloro-6-(3-methoxyphenyl)-1,3,5-triazin-2-amine

Prepared using 2,4-dichloro-6-(3′-methoxyphenyl)-1,3,5-triazine (0.379 g, 1.48 mmol), NaHCO₃ (0.249 g, 2.96 mmol) and 3-bromoaniline (0.16 ml, 1.48 mmol) to yield 0.429 g (74%) of a white solid; mp 177.5-177.7° C.(from toluene); HRMS (ESI) 391.0059 (M+H⁺. C₁₆H₁₃N₄O³⁵Cl⁷⁹Br requires 390.9961).

### 2.4 4-chloro-6-(3-methoxyphenyl)-N-(4-methyl-3-(morpholinosulfonyl)phenyl)-1,3,5-triazin-2-amine

Prepared using 2,4-dichloro-6-(3′-methoxyphenyl)-1,3,5-triazine (0.379 g, 1.48 mmol), NaHCO₃ (0.249 g, 2.96 mmol) and 4-methyl-3-(morpholine-4-sulfonyl)-aniline (0.379 g, 1.48 mmol) to yield 0.348 g (49%) of a white solid; mp 174.1-174.5° C. (from toluene); HRMS (ESI) 476.1213 (M+H⁺. C₂₁H₂₃N₅O₄ ³⁵ClS requires 476.1159).

General procedure for the preparation of 6-(3-methoxyphenyI)-N-aryl-[1,3,5]triazine-2,4-diamines.

35% Aqueous ammonia (1 ml) was added to a solution of 2-anilino-4-(3′-methoxyphenyI)-6-chloro-1,3,5-triazine (0.152 mmol) in 1,4-dioxane (2 ml) and the reaction mixture was slowly heated to 60° C. over 2 hours. The content of the flask was diluted with water (2 ml), extracted with diethyl ether (3×2 ml). Combined organic layers were dried over MgSO₄, the solvent was evaporated under reduced pressure and the residue was subjected to a flash column chromatography.

### 2.5 4-(4-amino-6-(3-methoxyphenyI)-1,3,5-triazin-2-ylamino)phenol

Prepared from 4-(4-chloro-6-(3-methoxyphenyl)-1,3,5-triazin-2-ylamino)phenol (50 mg, 0.152 mmol) to yield 33 mg (70%) of a light-yellow solid after column eluting with petroleum ether (40-60° C.):AcOEt=70:30. mp 93.5-94.0° C. (decamp.); HRMS (ESI) 310.1288 (M+H⁺. C₁₆H₁₆N₅O₂requires 310.1304).

### 2.6 6-(3-methoxyphenyl)-N2-(3-nitrophenyl)-1,3,5-triazine-2,4-diamine

Prepared from 4-chloro-6-(3-methoxyphenyl)-N-(3-nitrophenyl)-1,3,5-triazin-2-amine (54 mg, 0.152 mmol) to yield 35 mg (68%) of a light-yellow solid after column eluting with petroleum ether (40-60° C.):AcOEt=70:30; mp 196.9-197.2° C.; HRMS (ESI) 339.1213 (M+H⁺. C₁₆H₁₅N₆O₃ requires 339.1206).

### 2.7 6-(3-methoxyphenyl)-N2-methyl-N4-(3-nitrophenyl)-1,3,5-triazine-2,4-diamine

Prepared from 4-chloro-6-(3-methoxyphenyI)-N-(3-nitrophenyl)-1,3,5-triazin-2-amine (49 mg, 0.137 mmol), methylamine hydrochloride (14 mg, 0.206 mmol) and Na₂CO₃ to yield 39 mg (81%) of a light-yellow solid; mp 170.9-171.3° C. (from petroleum ether (40-60° C.)/AcOEt); HRMS (ESI) 353.1328 (M+H⁺. C₁₇H₁₇N₆O₃ requires 353.1362).

### 2.8 4-(hydroxyamino)-6-(3-methoxyphenyl)-N-(3-nitrophenyl)-1,3,5-triazin-2-amine

Prepared from 4-chloro-6-(3-methoxyphenyl)-N-(3-nitrophenyl)-1,3,5-triazin-2-amine (61 mg, 0.171 mmol), hydroxylamine hydrochloride (18 mg, 0.256 mmol), Na₂CO₃ (27 mg, 0.256 mmol) in 3 ml of DMF to yield 50 mg (83%) of a light-yellow solid; mp 197.1-197.5° C.; HRMS (ESI) 355.1166 (M+H⁺. C₁₆H₁₅N₆O₄ requires 355.1155).

### 2.9 N2-(3-bromophenyl)-6-(3-methoxyphenyl)-1,3,5-triazine-2,4-diamine

Prepared from N-(3-bromophenyl)-4-chloro-6-(3-methoxyphenyl)-1,3,5-triazin-2-amine (73 mg, 0.186 mmol) to yield 45 mg (65%) of a off-white solid after column eluting with petroleum ether (40-60° C.):AcOEt=70:30; mp 141.9-142.1° C.; HRMS (ESI) 372.0458 (M+H⁺. C₁₆H₁₅N₅O⁷⁹Br requires 372.0460).

### 2.10 6-(3-methoxyphenyl)-N2-(4-methyl-3-(morpholinosulfonyl)phenyl)-1,3,5-triazine-2,4-diamine

Prepared from 4-chloro-6-(3-methoxyphenyl)-N-(4-methyl-3-(morpholinosulfonyl)phenyl)-1,3,5-triazin-2-amine (76 mg, 0.160 mmol) to yield 35 mg (48%) of a white solid after column eluting with petroleum ether (40-60° C.):AcOEt=30:70; mp˜100° C. (decomp.); HRMS (ESI) 457.1691 (M+H⁺. C₂₁H₂₅N₆O₄S requires 457.1658).

### 3.2 tert-Butyl 5-(4-chloro-6-(3-nitrophenylamino)-1,3,5-triazin-2-yl)-4-phenylthiazol-2-yl(methyl)carbamate

A 1.8 M solution of LDA in THF (1.1 ml, 2.00 mmol, 1.1 eq) was added to a solution of a corresponding thiazole (0.528 g, 1.82 mmol, 1 eq) in dry THF (5 ml) cooled to −78° C. (dry ice—acetone bath). The reaction mixture was stirred for 45 min. In another flask, cyanuric chloride (0.402 g, 2.18 mmol, 1.2 eq) was dissolved in dry THF (5 ml) and the resulting solution was cooled to −78° C. The solution of formed anion was transferred via cannular to the content of the second flask and the reaction mixture was stirred for another 30 min, quenched with water (20 ml), extracted with ethyl acetate (3×30 ml), combined organic layers washed with brine (50 ml), dried over MgSO₄, evaporated under reduced pressure and the residue was subjected to a flash column chromatography eluting with petroleum ether (40-60° C.):AcOEt=95:5 to give 0.387 g (49%) of a yellow solid; mp 163° C. (decomp.); HRMS (ESI) 438.0739 (M+H⁺. C₁₈H₁₈N₅O₂³⁵Cl₂ requires 438.0558)

Prepared using the corresponding tert-butyl 5-(4,6-dichloro-1,3,5-triazin-2-yl)-4-phenylthiazol-2-yl(methyl)carbamate (98 mg, 0.224 mmol), NaHCO₃ (38 mg, 0.448 mmol) and 3-nitroaniline (31 mg, 0.224 mmol) to yield 60 mg (50%) of the desired compound as yellow solid after column eluting with petroleum ether (40-60° C.):AcOEt=85:15. HRMS (ESI) 540.1460 (M+H⁺. C₂₄H₂₃N₇O₄ ³⁵ClS requires 540.1221).

### 3.3 tert-Butyl5-(4-amino-6-(3-nitrophenylamino)-1,3,5-triazin-2-yl)-4-phenylthiazol-2-yl(methyl)carbamate

δ_{H} (400 MHz, CDCl₃) 8.34 (1H, br. s), 7.79 (1H, d, J 7.5, Ar), 7.73-7.70 (2H, m, Ar), 7.57 (1H, d, J 7.9, Ar), 7.35-7.25 (4H, m, Ar), 5.29 (2H, br. s, NH₂), 3.58 (3H, s, NCH₃), 1.61 (9H, s, C(CH₃)₃); δ_{C} (100 MHz, CDCl₃) 168.5, 166.4, 163.9, 162.2, 153.0, 148.4, 139.6, 135.7, 130.1, 128.4, 127.6, 125.3, 124.3, 117.3, 114.4, 83.7, 60.4, 33.8, 28.2.

### 3.4 6-(2-(methylamino)-4-phenylthiazol-5-yl)-N2-(3-nitrophenyl)-1,3,5-triazine-2,4-diamine

To a suspension of above tert-butyl5-(4-amino-6-(3-nitrophenylamino)-1,3,5-triazin-2-yl)-4-phenylthiazol-2-yl(methyl)carbamate (37 mg, 0.071 mmol) in 1 ml of DCM, TFA (1 ml) was added and the reaction mixture was stirred at room temperature for 24 hours. The solvents were evaporated under reduced pressure, the residue was neutralised with 5 ml of saturated Na₂CO₃ and extracted with ethyl acetate (3×5 ml). Combined organic layers were dried over MgSO₄, evaporated under reduced pressure and the residue was filtered through a plug of silica to give 30 mg (100%) of a yellow solid; mp 277° C.; HRMS (ESI) 421.1265 (M+H⁺. C₁₃H₁₇N₈O₂S requires 421.1195).

### Example 1a

### Preparation of a Compound of Formula IV

### 1.1a N-[4-Methyl-3-(morpholine-4-sulfonyl)-phenyl]-guanidine was Prepared as Follows:

2-Methyl-5-nitro-benzenesulfonyl chloride (20 mmol) dissolved in 20m1 THF was treated with morpholine (40 mmol) in the presence of triethylamine (25 mmol). After stirring for 2 hours at room temperature the reaction mixture was concentrated in vacuo to give 4-(2-methyl-5-nitro-benzenesulfonyl)-morpholine as a brown solid (95% yield), m.p. 114-115° C. MS (ESI⁺) m/z 287.82 (M+H)⁺.

To a mixture of the latter compound (7 mmol) in EtOH (10 ml) AcOH (5 ml) was added. The mixture was heated to 65° C. and Fe powder (28 mmol) was added portion wise. After refluxing for 1.5 hours the reaction mixture was cooled to room temperature, filtered and washed with a minima amount of EtOAc/EtOH. The filtrate was evaporated to dryness. The resulted precipitates were basified with excess aq NaOH. The aqueous phase was then extracted several times with EtOAc. The organic fractions were combined and evaporated to yield 4-methyl-3-(morpholine-4-sulfonyl)-phenylamine. Brown solid (39% yield), MS (ESI⁺) m/z 257.09 (M+H)⁺.

A mixture of 4-methyl-3-(morpholine-4-sulfonyl)-phenylamine (15 mmol) in EtOH (20 ml) was cooled on an ice bath and treated with HCl (1.3 ml, 37% solution in H₂O) followed by cyanamide (2.2 ml, 50% in H₂O, 60 mmol) was added dropwise, and the mixture heated at 100° C. for 17 hours. Upon completion of the reaction, the mixture was concentrated. The precipitate was washed with petroleum ether/EtOAc (4:1), filtered and dried to give N-[4-methyl-3-(morpholine-4-sulfonyl)-phenyl]-guanidine as a brown solid. ¹H NMR (DMSO-d₆) δ 2.43 (s, 3H, CH₃), 3.05 (s, 4H, CH₂×2), 3.63 (s, 4H, CH₂×2), 7.45 (m, 4H, NH₂, NH×2), 7.94 (d, 1H, J=8.0Hz, Ph-H), 8.17 (s, 1H, Ph-H), 8.28 (d, 1H, J=8.0 Hz, Ph-H). MS (ESI⁺) m/z 299.11 (M+H)⁺.

The following compounds were synthesised by an analogous route.

1-(3-Nitrophenyl)guanidine. ¹H NMR (DMSO-d₆): δ 7.70 (m, 5H, Ph-H×2, NH, NH₂), 8.09 (m, 1H, Ph-H), 8.19 (m, 1H, Ph-H). MS (ESI⁺) m/z 181.07 (M+H)⁺.

1-(3-Hydroxyphenyl)guanidine. ¹H NMR (DMSO-d₆) δ 6.82 (m, 2H, Ph-H×2), 7.02 (m, 2H, Ph-H×2), 7.30 (s, 4H, NH₂ & NH×2), 9.75 (s, 1H, OH). MS (ESI⁺) m/z 152.08 (M+H)⁺.

1-(4-Hydroxyphenyl)guanidine. ¹H NMR (DMSO-d₆): δ 6.63 (m, 2H, Ph-H×2), 6.70 (m, 1H, Ph-H), 7.20 (t, 1H, J=8.0 Hz, Ph-H), 7.44 (s, 4H, NH₂, NH×2), 9.80 (s, 1H, OH). MS (ESI⁺) m/z 152.31 (M+H)⁺.

1-(3-(Morpholine-4-carbonyl)phenyl)guanidine. ¹H NMR (DMSO-d₆) δ 3.61 (s, 8H, CH₂), 4.28 (m, 1H, NH), 7.24 (t, 1H, J=1.6 Hz, Ph-H), 7.31 (t, 1H, J=2 Hz, Ph-H), 7.33 (t, 1H, J=2.4 Hz, Ph-H), 7.49 (t, 1H, J=8 Hz, Ph-H), 7.57 (s, 2H, NH₂), 10.00 (s, 1H, NH). MS (ESI⁺) m/z 249.10 (M+H)⁺.

4-Guanidinobenzenesulfonamide. ¹H NMR (DMSO-d₆, 400 MHz): δ 6.80 (d, 1H, J=8.4 Hz, NH), 7.24 (s, 1H, NH), 7.39 (d, 2H, J=8.8 Hz, Ph-H), 7.39 (d, 2H, J=8.4 Hz, Ph-H), 7.77 (s, 2H, NH₂). MS (ESI⁺) m/z 215.07 (M+H)⁺.

3-Guanidinobenzenesulfonamide. ¹H NMR (DMSO-d₆, 400 MHz): δ 7.46 (m, 3H, NH & NH₂), 7.63 (t, 1H, J=8.0 Hz, Ph-H), 7.65 (m, 1H, Ph-H), 7.69 (m, 2H, Ph-H×2), 7.72 (s, 2H, NH₂), 10.36 (s, 1H, NH). MS (ESI⁺) m/z 215.06 (M+H)⁺.

Biological Activity

Kinase assays. The compounds from the examples above were investigated for their ability to inhibit the enzymatic activity of various protein kinases using the method as disclosed in Wang, S. et al. J Med Chem 2004, 47, 1662. This was achieved by measurement of incorporation of radioactive phosphate from ATP into appropriate polypeptide substrates. Recombinant protein kinases and kinase complexes were produced or obtained commercially. Assays were performed using 96-well plates and appropriate assay buffers (typically 25 mM β-glycerophosphate, 20 mM MOPS, 5 mM EGTA, 1 mM DTT, 1 mM Na₃VO₃, pH 7.4), into which were added 2-4 μg of active enzyme with appropriate substrates. The reactions were initiated by addition of Mg/ATP mix (15 mM MgCl₂+100 μM ATP with 30-50 kBq per well of [γ-³²P]-ATP) and mixtures incubated as required at 30° C.; Reactions were stopped on ice, followed by filtration through p81 filterplates or GF/C filterplates (Whatman Polyfiltronics, Kent, UK). After washing 3 times with 75 mM aq orthophosphoric acid, plates were dried, scintillant added and incorporated radioactivity measured in a scintillation counter (TopCount, Packard Instruments, Pangbourne, Berks, UK). Compounds for kinase assay were made up as 10 mM stocks in DMSO and diluted into 10% DMSO in assay buffer. Data was analysed using curve-fitting software (GraphPad Prism version 3.00 for Windows, GraphPad Software, San Diego Calif. USA) to determine IC₅₀ values (concentration of test compound which inhibits kinase activity by 50%).

MTT cytotoxicity assay. The compounds from the examples above were subjected to a standard cellular proliferation assay using the method described previously (Haselsberger, K. et al. Anti Cancer Drugs 1996, 7, (3), 331-8. Loveland, B. E. et al. Biochemistry International 1992, 27, (3), 501-10). Human tumour cell lines were obtained from ECACC (European Collection of Cell Cultures). Standard 72-h MTT (thiazolyl blue; 3-[4,5-dimethylthiazol-2-yl]-2,5-diphenyltetrazolium bromide, 2 mg/ml in phosphate buffered saline) assays were performed. In short: cells were seeded into 96-well plates according to doubling time and incubated overnight at 37° C. Test compounds were made up in DMSO and a ⅓ dilution series prepared in 100 μL cell media, added to cells (in triplicates) and incubated for 72 hr at 37° C. MTT was made up as a stock of 5 mg/mL in cell media and filter-sterilised. Media was removed from cells followed by a wash with 200 μL PBS. MTT solution was then added at 20 μL per well and incubated in the dark at 37° C. for 4 h. MTT solution was removed and cells again washed with 200 μL PBS. MTT dye was solubilised with 200 μL per well of DMSO with agitation. Absorbance was read at 550 nm on an Anthos Labtec Systems plate reader. The data analysis used program Deltasoft 3™ and Microsoft Excel to determine IO₅₀ or GI₅₀ values (concentration of test compound which inhibits cell growth by 50%).

CLL apoptosis assay. Compounds were thawed on ice and aliquotted to 0.5 ml microcentrifuge tubes and stored at −20′C to avoid multiple freeze-thaw cycles. Compound aliquots were thawed on ice and diluted as required in sterile PBS immediately prior to drugging experiment. Primary CLL cells were isolated from ACD whole blood using standard Ficoll (Ficoll-Paque Plus,GE Healthcare) separation and selected for B cells with RosetteSep B cell enrichment cocktail (StemCell Tech.). Cells were incubated at 1E6-3E6 cells/ml in RPMI 1640 plus 10% human serum and antibiotics at 37′C in 24 well plates. Inhibitor compounds were added at time 0 and a sample was maintained with media vehicle only. At 24 hours, cells were transferred to a 12×75 tube for Annexin-PI viability assay. Cells were centrifuged at 1500 rpm for 5 minutes then incubated at RT in dark for 30 minutes with appropriate reagent plus binding buffer with calcium. After incubation, 800 ul of binding buffer was added for flow cytometry analysis on the EPICS-XL (Beckman-Coulter).

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the claims.

### Example A1

Biological activity of the example compounds is summarized in Tables A1 and A2.

TABLE A1

| | Kinase inhibition | | | Cytotoxicity | | |
|---|---|---|---|---|---|---|
| | IC₅₀ μM | | | GI₅₀ μM | | |
| | CDK2-cyclinE | CDK7-cyclinH | CDK9-cyclinT1 | HCT116 | MCF7 | MRC5 |
| 1.1 | 0.232 | >1 | 0.041 | 0.06 | 0.06 | 3.14 |
| 1.2 | | | | 0.40 | 0.23 | 1.05 |
| 1.3 | 0.045 | | 0.019 | 0.03 | 0.07 | 0.54 |
| 1.4 | 0.147 | 3.741 | 0.085 | 0.59 | 0.82 | 4.43 |
| 1.5 | 0.052 | | 0.033 | 5.88 | 4.96 | 79.92 |
| 1.6 | | | | 0.55 | 1.87 | >100 |
| 1.8 | | | | 0.34 | 1.64 | 25.99 |
| 1.7 | | | | 0.05 | 0.08 | 0.26 |
| 2.4 | | | | 0.53 | 1.58 | 0.58 |
| 2.6 | | | | 7.09 | 7.07 | 37.60 |
| 2.8 | | | | 3.48 | 6.42 | 4.24 |
| 2.13 | | | | 3.32 | 3.77 | 4.33 |

TABLE A2
anti-proliferative activity of Example 1.1

| human cell line | | 72-h MTT |
|---|---|---|
| origin | designation | IC₅₀ (μM) |
| breast | MCF-7 | 0.060 |
| | MDA-MB-231 | 0.923 |
| cervix | HeLa | 0.661 |
| | HCT-116 | 0.060 |
| lung | A549 | 0.534 |
| | NCI-H460 | 0.681 |
| ovarian carcinoma | A2780 | 0.147 |
| pancreatic carcinoma | PANC W1 | 0.366 |
| | Mia-Paca-2 | 0.468 |
| mean | | 0.433 |

1. A compound of formula I or a pharmaceutically acceptable salt or solvate thereof: 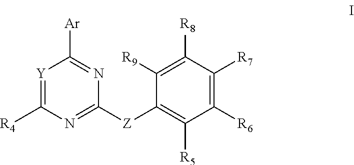
wherein:
Ar is a 5-membered heteroaryl ring comprising one or two heteroatoms wherein heteroatoms are independently selected from S, O, N, and Se, and wherein Ar is optionally substituted by R¹ and R²;
Z is NH, NHCO, NHSO₂, N-alkyl, CH₂NH, CH₂N-alkyl, CH₂, CH₂CH₂, CH═CH, CH₂CONH, SO₂, or SO;
Y is CR³;
R¹, R², R⁵, R⁶, R⁷, R⁸ and R⁹ are each independently H, alkyl, or R¹³
R¹³ is selected from R¹⁰, alkyl-R¹⁰, aryl, heteroaryl and combinations of two or more thereof and combinations with one or more alkyl and R¹¹, or R¹³ is one or more moieties R¹⁴ selected from O-, N-, NH-, CO-, COO-, CON-, CONH-, SO₂-, SO₂N-, SO₂NH-linking one or more alkyl, aryl, heteroaryl or R¹⁰ or R¹¹ groups or combinations thereof, directly or via a moiety selected from alkylene, arylene, heteroarylene or combinations thereof, wherein alkyl, aryl, heteroaryl groups or moieties thereof may be substituted with one or more groups R¹⁵ selected from halogeno, NH₂, NO₂, CN, OH, COOH, CONH₂, C(═NH)NH₂, SO₃H, SO₂NH₂, SO₂CH₃, OCH₃, CF₃ or R¹³ is selected from a group R¹⁵;
or two of R⁵ to R⁹ are linked to form a cyclic ether containing one or more oxygen atoms;
R³ is selected from alkyl and R¹³ as hereinbefore defined, with the proviso that when Ar is a 5-membered heterocycle comprising one or two N heteroatoms and Z is NH, then R³ is selected from C₃₊ alkyl and R¹³;
R⁴ is selected from H, alkyl and R¹³;
wherein at least one of R¹, R², R⁴, R⁵, R⁶, R⁷, R⁸ and R⁹ and R³ or R¹² where present, comprises a group R¹⁰ or R¹¹ wherein R¹⁰ and R¹¹ comprise one or more solubilising moieties chosen from i) neutral hydrophilic groups, ii) ionisable organic acids, iii) ionisable organic bases and combinations thereof.

2-18. (canceled)

19. A compound according to claim 1 wherein at least one R¹⁰ or R¹¹ further comprises an immobilising moiety chosen from chemical functions or moieties providing covalent or non-covalent attachment or binding to a solid phase or an immobile receptor.

20. A compound according to claim 1 wherein Ar comprises two heteroatoms, one of which is S and the other of which is N.

21. A compound according to claim 1 which is of formula I′ 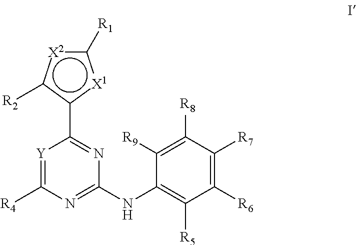
wherein X¹ and X² are each independently selected from NH or N, O, S, Se, CH and CR¹⁵, with the proviso that at least one of X¹ and X² is selected from NH or N, O, S and Se.

22. A compound according to claim 21 wherein:
one of X¹ and X² is CH or CR¹⁵, and the other of X¹ and X² is S, O, NH, NR¹⁵, or Se; or
one of X¹ and X² is S, O or Se, and the other of X¹ and X² is N; or
one of X¹ and X² is N, and the other of X¹ and X² is NH or NR¹⁵.

23. A compound according to claim 1 wherein:
when R¹⁰ or R¹¹ comprises a neutral hydrophilic group (i), the group comprises a mono-, di- and polyhydroxylated saturated or unsaturated aliphatic, alicyclic or aromatic systems, carbohydrate derivatives, ethers and polyethers optionally containing one or more hydroxyl groups, O- and/or S-containing heterocyclic systems optionally containing one or more hydroxyl groups, aliphatic or aromatic systems containing a carboxamide, sulfoxide, sulfone, or sulfonamide function, and halogenated alkylcarbonyl groups; or
when R¹⁰ or R¹¹ comprise an ionisable organic acid (ii) comprising one or more of the functional groups COOH, SO₃H, OSO₃H, PO₃H₂, and OPO₃H₂; or
where R¹⁰ or R¹¹ comprises an ionisable basic group (iii) as hereinbefore defined, this preferably includes aliphatic, alicyclic, aromatic, or heterocyclic groups comprising one or more of the functions —O—, —NH₂, —NH—, ═N—, quarternary amine salts, guanidine, and amidine, optionally substituted by one or more substituents selected from halogen, SO₂alkyl, alkyl optionally substituted by one or more OH or halogen groups, CHO, COalkyl, aralkyl, COOalkyl and an ether group substituted by one or more OH groups.

24. A compound according to claim 1 wherein R¹⁰ and R¹¹ consist of natural or unnatural amino acid residues and peptides, or their derivatives; or
R¹⁰ or R¹¹ is selected from the group consisting of:
i) OSO₃H, PO₃H₂, OPO₃H₂;
ii) Y′, where Y′ is selected from aliphatic, alicyclic, aromatic, or heterocyclic groups comprising one or more of the functions —O—, —NH₂, —NH—, ═N—, amidine, optionally substituted by one or more substituents selected from halogen, SO₂alkyl, alkyl optionally substituted by one or more OH or halogen groups, COalkyl, aralkyl, COOalkyl and an ether group substituted by one or more OH groups;
(iii) NHCO(CH₂)ₘ[NHCO(CH₂)_{m′}]ₚ[NHCO(CH₂)_{m′}]_{q}Y′ or NHCO(CH₂)ₜNH(CH₂)_{t′}Y′ where p and q are each 0 or 1, and m, m′, m″, t and t′ are each independently an integer from 1 to 10;
(iv) (CH₂)ₙNR¹⁹COR¹⁷, (CH₂)_{n′}NR²⁰SO₂R¹⁸, or SO₂R²¹, where R¹⁷, R¹⁸ and R²¹ are each alkyl groups optionally comprising one or more heteroatoms, and which are optionally substituted by one or more substituents selected from OH, NH₂, halogen and NO₂, R¹⁹ and R²⁰ are each independently H or alkyl, and n and n′ are each independently 0, 1, 2, or 3;
(v) an ether or polyether optionally substituted by one or more hydroxyl groups or one or more Y′ groups;
(vi) (CH₂)ᵣNH₂; where r is 0, 1 , 2, or 3;
(vii) (CH₂)_{r′}OH; where r′ is 0, 1, 2, or 3;
(viii) (CH₂)_{n″}NR²²COR²³ where R²² is H or alkyl, n″ is 0, 1, 2 or 3 and R²³ is an aryl or heteroaryl group, each of which may be optionally substituted by one or more substituents selected from halogeno, NO₂, OH, alkoxy, NH₂, COOH, CONH₂ and CF₃;
(ix) SO₂NR²⁴R²⁵ where R²⁴ and R²⁵ are each independently H, alkyl, aralkyl, CO-alkyl or aryl, with the proviso that at least one of R²⁴ and R²⁵ is other than H, or R²⁴ and R²⁵ are linked to form a cyclic group optionally containing one or more heteroatoms selected from N, O and S, and wherein said alkyl, aryl or cyclic group is optionally substituted by one or more substituents selected from halogeno, NO₂, OH, alkoxy, aryl, NH₂, COON, CH₂CO₂-alkyl, CONH₂ and CF₃; and
(x) N-piperidinyl, piperidinyl, N-piperazinyl, N-diazepanyl, N-pyridinyl, N-pyrrolidinyl, N-morpholinyl or N-thiomorpholinyl, each of which may be optionally substituted by one or more alkyl, alkoxy, aryl, CHO or CO-alkyl groups.

25. A compound according to claim 1 wherein each R¹⁰ or R¹¹ is independently selected from a C₁₋₃₀ hydrocarbyl group, optionally comprising up to twelve heteroatoms selected from N, S, and O, and optionally bearing up to six substituents each independently selected from a group R¹⁵ or comprising a moiety R¹⁴ and a group R¹⁵.

26. A compound according to claim 1, wherein R³ is selected from CN, CF₃, halogeno, NO₂, NH₂, NH-alkyl, N-(alkyl)(R¹⁰), NH-cycloheteroalkyl, NHSO₂R¹⁰, CONH₂, CONH-(alkyl). CON-(alkyl)(R¹⁰), R¹⁰, CO-cycloheteroalkyl, CO-heteroaryl, CONH-heteroaryl, CH₂-cycloheteroalkyl, CH₂-heteroaryl, cycloheteroalkyl, heteroaryl, and C₂₋₆ or C₄₋₆ alkyl, wherein alkyl, cycloheteroalkyl, aryl, aralkyl, heteroaryl groups may be further substituted with one or more groups selected from halogeno, NO₂, CN OH, O-methyl, NH₂, COOH, CONH₂ and CF₃.

27. A compound according to claim 1, wherein R³ is CN or halogeno.

28. A compound of formula I′: 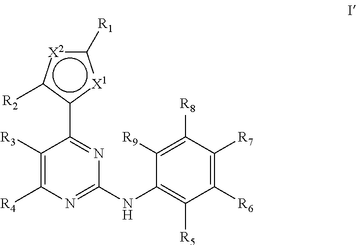
selected from the group of compounds as shown in Table 1 wherein X¹═S, X²═N

| Cpd | R₁ | R₂ | R₃ | R₄ | R₅ | R₆ | R₇ | R₈ | R₉ |
|---|---|---|---|---|---|---|---|---|---|
| 1.1 | NHCH₃ | CH₃ | CN | H | H | “MS” | CH₃ | H | H |
| 1.2 | NHCH₃ | CH₃ | CN | H | H | H | OH | H | H |
| 1.3 | NHCH₃ | CH₃ | CN | H | H | OH | H | H | H |
| 1.4 | NHCH₃ | CH₃ | CN | H | H | “MC” | H | H | H |
| 1.5 | NHCH₃ | CH₃ | CN | H | H | “AcPzC” | H | H | H |
| 1.6 | NHCH₃ | CH₃ | CN | H | H | COOH | H | H | H |
| 1.7 | NHCH₃ | CH₃ | CN | H | H | NO₂ | H | H | H |
| 1.8 | NHCH₃ | CH₃ | CN | H | H | H | SO₂NH₂ | H | H |
| 1.9 | NHCH₃ | CH₃ | CN | H | H | SO₂NH₂ | H | H | H |
| 1.10 | NHCH₃ | CH₃ | CN | H | H | “MePzC” | H | H | H |
| 1.11 | NHCH₃ | CH₃ | CN | H | H | H | “M” | H | H |
| 1.12 | NHCH₃ | CH₃ | CN | H | H | “MS” | H | H | H |
| 1.13 | NHCH₃ | CH₃ | CN | H | H | H | “MS” | H | H |
| 1.14 | NHCH₃ | CH₃ | CN | H | H | SO₂CH₃ | H | H | H |
| 1.15 | NHCH₃ | CH₃ | CN | H | H | “PzC” | H | H | H |
| 1.16 | NH₂ | CH₃ | CN | H | H | “MS” | CH₃ | H | H |
| 1.17 | NH₂ | CH₃ | CN | H | H | H | OH | H | H |
| 1.18 | NH₂ | CH₃ | CN | H | H | OH | H | H | H |
| 1.19 | NH₂ | CH₃ | CN | H | CH₃ | H | OH | CH₃ | H |
| 1.20 | NH₂ | CH₃ | CN | H | H | “MC” | H | H | H |
| 1.21 | NH₂ | CH₃ | CN | H | H | “MePzC” | H | H | H |
| 1.22 | NH₂ | CH₃ | CN | H | H | “AcPzC” | H | H | H |
| 1.23 | NH₂ | CH₃ | CN | H | H | “PzC” | H | H | H |
| 1.24 | NH₂ | H | CN | H | H | “MS” | CH₃ | H | H |
| 1.27 | NHCH₃ | CH₃ | CN | H | H | “BPzC” | H | H | H |
| 1.28 | NH₂ | CH₃ | CN | H | H | “BPzC” | H | H | H |
| 1.29 | NH₂ | CH₃ | CN | H | H | “MePdCB” | H | H | H |
| 1.30 | NHCH₃ | CH₃ | CN | H | H | “MePdCB” | H | H | H |
| 1.37 | “PyMeA” | CH₃ | CN | H | H | “MePzC” | H | H | H |
| 1.38 | “PyMeA” | CH₃ | CN | H | H | “PzC” | H | H | H |
| 1.39 | NH(CH₂)₂CH₃ | CH₃ | CN | H | H | “PzC” | H | H | H |
| 1.40 | NHCH₃ | CH₃ | CN | H | H | (2-hydroxyethyl)“PzC” | H | H | H |
| 1.41 | NHCH₃ | CH₃ | CN | H | H | (2-methoxyethyl)“PzC” | H | H | H |
| 1.42 | NH₂ | CH₃ | CN | H | H | (2-methoxyethyl)“PzC” | H | H | H |
| 1.43 | NH(CH₂)₂CH₃ | CH₃ | CN | H | H | (2-methoxyethyl)“PzC” | H | H | H |
| 1.44 | NH(CH₂)₂CH₃ | CH₃ | CN | H | H | (2-methoxyethyl)“PdC” | H | H | H |
| 1.45 | NH(CH₂)₂CH₃ | CH₃ | CN | H | H | H | “MeDz” | H | H |
| 1.46 | NHCH₂CH₃ | CH₃ | CN | H | H | H | “MeDz” | H | H |
| 1.47 | NHCH₃ | CH₃ | CN | H | H | H | “MeDz” | H | H |
| 1.48 | NH₂ | CH₃ | CN | H | H | H | “MeDz” | H | H |
| 1.49 | NH₂ | CH₃ | CN | H | H | “MeDz” | H | H | H |
| 1.50 | NHCH₃ | CH₃ | CN | H | H | “MeDz” | H | H | H |
| 1.51 | NHCH₂CH₃ | CH₃ | CN | H | H | “MeDz” | H | H | H |
| 1.52 | NH(CH₂)₂CH₃ | CH₃ | CN | H | H | “MeDz” | H | H | H |
| 1.53 | NHCH₃ | CH₃ | CN | H | CH₃ | H | OH | CH₃ | H |

and wherein
MS=morpholine-4-sulfonyl 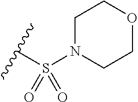
PzC=piperazine-1-carbonyl or piperazin-1-ylmethanone 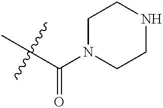
AcPzC=4-Acetylpiperazine-1-carbonyl 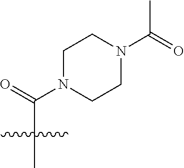
MePzC=4-methylpiperazine-1-carbonyl or 4-methylpiperazin-1-ylmethanone 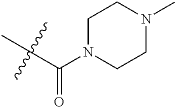
M=morpholino 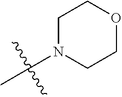
MC=morpholin-4-carbonyl or morpholin-4-yl-methanone 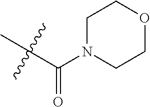
BPzC—benzylpiperazine-1-carbonyl 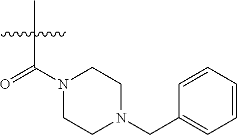
MePdCB—4-(1-methylpiperidine-4carbonyl)benzoyl 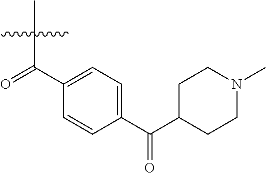
MeDz=4-methyl-1,4-diazepan-1-yl 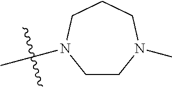
PyEtA=2-(pyridine-3-yl)ethylamino 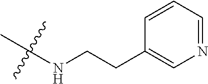
PyMeA=pyridin-3-ylmethylamino

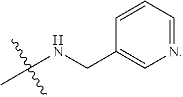

29. A process for the preparation of a compound formula I according to claim 1, comprising:
(1) reacting a compound of formula III 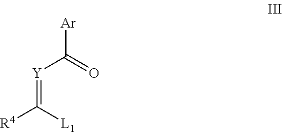
where L¹ is a leaving group, with a compound of formula IV 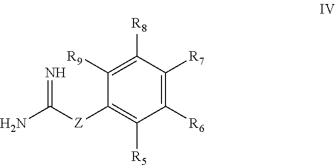
or (2) reacting a compound of formula XI 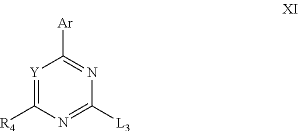
where Y is CR³, L³ is any leaving group, preferably a halogeno group, with a compound of formula XII

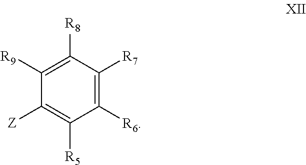

30. A process for the preparation of a compound of formula I′ according to claim 21, comprising:
(1) the condensation reaction between a compound of formula VII′ 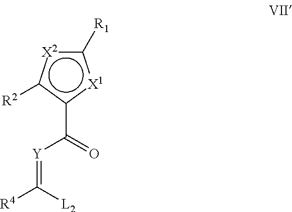
Where L₂ is a leaving group; with a phenylguanidine of formula VIII′ 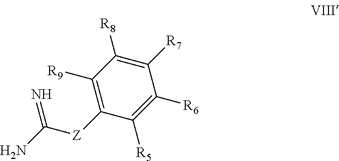
or (2) condensation reaction of a compound of formula XII′ 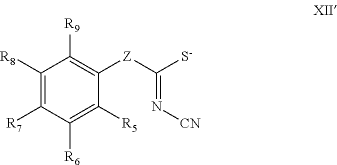
with an amidine of formula XIII′ 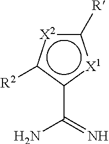
in the presence of base.

31. A pharmaceutical composition comprising a compound of formula I, or a pharmaceutically acceptable salt or solvate thereof, according to claim 1, and one or more diluents, carriers or excipients.

32. A method for treating a condition mediated by one or more enzymes selected from CDK, aurora kinase, GSK, PLK, BCR-ABL, FLT, IKK, JAK, PDGF or VEGF and Src family enzymes, in a human or animal subject, comprising administering to said human or animal in need thereof a therapeutically effective amount of a compound of formula I or a pharmaceutically acceptable salt or solvate thereof according to claim 1.

33. A method according to claim 32, wherein the condition is mediated by CDK2, CDK7, CDK8, CDK9, CDK11, GSK-3, aurora kinase, PLK or tyrosine kinase enzyme.

34. A method of treating of treating a proliferative disorder in a human or animal in need of such treatment, said method comprising administering to said human or animal a therapeutically effective amount of a compound of formula I or a pharmaceutically acceptable salt or solvate thereof according to claim 31.

35. A method according to claim 34, wherein said proliferative disorder is selected from cancers, leukaemias and other disorders associated with uncontrolled cellular proliferation, a viral disorder, a cardiovascular disease, a CNS disorder, an autoimmune disease, a bone disease, a hormone-related disease, a metabolic disorder, stroke, alopecia, an inflammatory disease or an infectious disease.

36. A method according to claim 35, wherein the proliferative disorder is a cancer or leukaemia.

37. A method according to claim 35, wherein the proliferative disorder is a neoplasm selected from the group consisting of chronic lymphocytic leukaemia, lymphoma, leukaemia, breast cancer, lung cancer, prostate cancer, colon cancer, melanoma, pancreatic cancer, ovarian cancer, squamous carcinoma, carcinoma of head and neck, endometrial cancer, and oesophageal carcinoma.

38. A method according to claim 35, wherein the proliferative disorder is psoriasis or restenosis.
